SEQUENCE LISTING (6 sequences)

SEQ ID NO: 1 - DNA, 6030 bp, homo sapiens
  CDS (32)..(751)
gttggcccccgttacttttcctctgggaaatatggcgcacgctgggagaacagggtacgataaccgggagatagtgatgaagtacatccattataagctgtcgcagaggggctacgagtgggatgcgggagatgtgggcgccgcgcccccgggggccgcccccgcgccgggcatcttctcctcgcagcccgggcacacgccccatacagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccaggcagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtgaagtcaacatgcctgccccaaacaaatatgcaaaaggttcactaaagcagtagaaataatatgcattgtcagtgatgttccatgaaacaaagctgcaggctgtttaagaaaaaataacacacatataaacatcacacacacagacagacacacacacacacaacaattaacagtcttcaggcaaaacgtcgaatcagctatttactgccaaagggaaatatcatttattttttacattattaagaaaaaaagatttatttatttaagacagtcccatcaaaactcctgtctttggaaatccgaccactaattgccaagcaccgcttcgtgtggctccacctggatgttctgtgcctgtaaacatagattcgctttccatgttgttggccggatcaccatctgaagagcagacggatggaaaaaggacctgatcattggggaagctggctttctggctgctggaggctggggagaaggtgttcattcacttgcatttctttgccctgggggctgtgatattaacagagggagggttcctgtggggggaagtccatgcctccctggcctgaagaagagactctttgcatatgactcacatgatgcatacctggtgggaggaaaagagttgggaacttcagatggacctagtacccactgagatttccacgccgaaggacagcgatgggaaaaatgcccttaaatcataggaaagtatttttttaagctaccaattgtgccgagaaaagcattttagcaatttatacaatatcatccagtaccttaagccctgattgtgtatattcatatattttggatacgcaccccccaactcccaatactggctctgtctgagtaagaaacagaatcctctggaacttgaggaagtgaacatttcggtgacttccgcatcaggaaggctagagttacccagagcatcaggccgccacaagtgcctgcttttaggagaccgaagtccgcagaacctgcctgtgtcccagcttggaggcctggtcctggaactgagccggggccctcactggcctcctccagggatgatcaacagggcagtgtggtctccgaatgtctggaagctgatggagctcagaattccactgtcaagaaagagcagtagaggggtgtggctgggcctgtcaccctggggccctccaggtaggcccgttttcacgtggagcatgggagccacgacccttcttaagacatgtatcactgtagagggaaggaacagaggccctgggcccttcctatcagaaggacatggtgaaggctgggaacgtgaggagaggcaatggccacggcccattttggctgtagcacatggcacgttggctgtgtggccttggcccacctgtgagtttaaagcaaggctttaaatgactttggagagggtcacaaatcctaaaagaagcattgaagtgaggtgtcatggattaattgacccctgtctatggaattacatgtaaaacattatcttgtcactgtagtttggttttatttgaaaacctgacaaaaaaaaagttccaggtgtggaatatgggggttatctgtacatcctggggcattaaaaaaaaaatcaatggtggggaactataaagaagtaacaaaagaagtgacatcttcagcaaataaactaggaaatttttttttcttccagtttagaatcagccttgaaacattgatggaataactctgtggcattattgcattatataccatttatctgtattaactttggaatgtactctgttcaatgtttaatgctgtggttgatatttcgaaagctgctttaaaaaaatacatgcatctcagcgtttttttgtttttaattgtatttagttatggcctatacactatttgtgagcaaaggtgatcgttttctgtttgagatttttatctcttgattcttcaaaagcattctgagaaggtgagataagccctgagtctcagctacctaagaaaaacctggatgtcactggccactgaggagctttgtttcaaccaagtcatgtgcatttccacgtcaacagaattgtttattgtgacagttatatctgttgtccctttgaccttgtttcttgaaggtttcctcgtccctgggcaattccgcatttaattcatggtattcaggattacatgcatgtttggttaaacccatgagattcattcagttaaaaatccagatggcaaatgaccagcagattcaaatctatggtggtttgacctttagagagttgctttacgtggcctgtttcaacacagacccacccagagccctcctgccctccttccgcgggggctttctcatggctgtccttcagggtcttcctgaaatgcagtggtgcttacgctccaccaagaaagcaggaaacctgtggtatgaagccagacctccccggcgggcctcagggaacagaatgatcagacctttgaatgattctaatttttaagcaaaatattattttatgaaaggtttacattgtcaaagtgatgaatatggaatatccaatcctgtgctgctatcctgccaaaatcattttaatggagtcagtttgcagtatgctccacgtggtaagatcctccaagctgctttagaagtaacaatgaagaacgtggacgcttttaatataaagcctgttttgtcttctgttgttgttcaaacgggattcacagagtatttgaaaaatgtatatatattaagaggtcacgggggctaattgctggctggctgccttttgctgtggggttttgttacctggttttaataacagtaaatgtgcccagcctcttggccccagaactgtacagtattgtggctgcacttgctctaagagtagttgatgttgcattttccttattgttaaaaacatgttagaagcaatgaatgtatataaaagcctcaactagtcatttttttctcctcttcttttttttcattatatctaattattttgcagttgggcaacagagaaccatccctattttgtattgaagagggattcacatctgcatcttaactgctctttatgaatgaaaaaacagtcctctgtatgtactcctctttacactggccagggtcagagttaaatagagtatatgcactttccaaattggggacaagggctctaaaaaaagccccaaaaggagaagaacatctgagaacctcctcggccctcccagtccctcgctgcacaaatactccgcaagagaggccagaatgacagctgacagggtctatggccatcgggtcgtctccgaagatttggcaggggcagaaaactctggcaggcttaagatttggaataaagtcacagaatcaaggaagcacctcaatttagttcaaacaagacgccaacattctctccacagctcacttacctctctgtgttcagatgtggccttccatttatatgtgatctttgttttattagtaaatgcttatcatctaaagatgtagctctggcccagtgggaaaaattaggaagtgattataaatcgagaggagttataataatcaagattaaatgtaaataatcagggcaatcccaacacatgtctagctttcacctccaggatctattgagtgaacagaattgcaaatagtctctatttgtaattgaacttatcctaaaacaaatagtttataaatgtgaacttaaactctaattaattccaactgtacttttaaggcagtggctgtttttagactttcttatcacttatagttagtaatgtacacctactctatcagagaaaaacaggaaaggctcgaaatacaagccattctaaggaaattagggagtcagttgaaattctattctgatcttattctgtggtgtcttttgcagcccagacaaatgtggttacacactttttaagaaatacaattctacattgtcaagcttatgaaggttccaatcagatctttattgttattcaatttggatctttcagggattttttttttaaattattatgggacaaaggacatttgttggaggggtgggagggaggaacaatttttaaatataaaacattcccaagtttggatcagggagttggaagttttcagaataaccagaactaagggtatgaaggacctgtattggggtcgatgtgatgcctctgcgaagaaccttgtgtgacaaatgagaaacattttgaagtttgtggtacgacctttagattccagagacatcagcatggctcaaagtgcagctccgtttggcagtgcaatggtataaatttcaagctggatatgtctaatgggtatttaaacaataaatgtgcagttttaactaacaggatatttaatgacaaccttctggttggtagggacatctgtttctaaatgtttattatgtacaatacagaaaaaaattttataaaattaagcaatgtgaaactgaattggagagtgataatacaagtcctttagtcttacccagtgaatcattctgttccatgtctttggacaaccatgaccttggacaatcatgaaatatgcatctcactggatgcaaagaaaatcagatggagcatgaatggtactgtaccggttcatctggactgccccagaaaaataacttcaagcaaacatcctatcaacaacaaggttgttctgcataccaagctgagcacagaagatgggaacactggtggaggatggaaaggctcgctcaatcaagaaaattctgagactattaataaataagactgtagtgtagatactgagtaaatccatgcacctaaaccttttggaaaatctgccgtgggccctccagatagctcatttcattaagtttttccctccaaggtagaatttgcaagagtgacagtggattgcatttcttttggggaagctttcttttggtggttttgtttattataccttcttaagttttcaaccaaggtttgcttttgttttgagttactggggttatttttgttttaaataaaaataagtgtacaataagtgtttttgtattgaaagcttttgttatcaagattttcatacttttaccttccatggctctttttaagattgatacttttaagaggtggctgatattctgcaacactgtacacataaaaaatacggtaaggatactttacatggttaaggtaaagtaagtctccagttggccaccattagctataatggcactttgtttgtgttgttggaaaaagtcacattgccattaaactttccttgtctgtctagttaatattgtgaagaaaaataaagtacagtgtgagatactg

SEQ ID NO: 2 - protein, 239 aa, homo sapiens
MAHAGRTGYDNREIVMKYIHYKLSQRGYEWDAGDVGAAPPGAAPAPGIFSSQPGHTPHTAASRDPVARTSPLQTPAAPGAAAGPALSPVPPVVHLTLRQAGDDFSRRYRRDFAEMSRQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 3 - DNA, 3035 bp, homo sapiens
  CDS (15)..(1313)
agagccagagcaggatggagaggagacgcatcacctccgctgctcgccgctcctacgtctcctcaggggagatgatggtggggggcctggctcctggccgccgtctgggtcctggcacccgcctctccctggctcgaatgccccctccactcccgacccgggtggatttctccctggctggggcactcaatgctggcttcaaggagacccgggccagtgagcgggcagagatgatggagctcaatgaccgctttgccagctacatcgagaaggttcgcttcctggaacagcaaaacaaggcgctggctgctgagctgaaccagctgcgggccaaggagcccaccaagctggcagacgtctaccaggctgagctgcgagagctgcggctgcggctcgatcaactcaccgccaacagcgcccggctggaggttgagagggacaatctggcacaggacctggccactgtgaggcagaagctccaggatgaaaccaacctgaggctggaagccgagaacaacctggctgcctatagacaggaagcagatgaagccaccctggcccgtctggatctggagaggaagattgagtcgctggaggaggagatccggttcttgaggaagatccacgaggaggaggttcgggaactccaggagcagctggcccgacagcaggtccatgtggagcttgacgtggccaagccagacctcaccgcagccctgaaagagatccgcacgcagtatgaggcaatggcgtccagcaacatgcatgaagccgaagagtggtaccgctccaagtttgcagacctgacagacgctgctgcccgcaacgcggagctgctccgccaggccaagcacgaagccaacgactaccggcgccagttgcagtccttgacctgcgacctggagtctctgcgcggcacgaacgagtccctggagaggcagatgcgcgagcaggaggagcggcacgtgcgggaggcggccagttatcaggaggcgctggcgcggctggaggaagaggggcagagcctcaaggacgagatggcccgccacttgcaggagtaccaggacctgctcaatgtcaagctggccctggacatcgagatcgccacctacaggaagctgctagagggcgaggagaaccggatcaccattcccgtgcagaccttctccaacctgcagattcgagaaaccagcctggacaccaagtctgtgtcagaaggccacctcaagaggaacatcgtggtgaagaccgtggagatgcgggatggagaggtcattaaggagtccaagcaggagcacaaggatgtgatgtgaggcaggacccacctggtggcctctgccccgtctcatgaggggcccgagcagaagcaggatagttgctccgcctctgctggcacatttccccagacctgagctccccaccaccccagctgctcccctccctcctctgtccctaggtcagcttgctgccctaggctccgtcagtatcaggcctgccagacggcacccacccagcacccagcaactccaactaacaagaaactcacccccaaggggcagtctggaggggcatggccagcagcttgcgttagaatgaggaggaaggagagaaggggaggagggcggggggcacctactacatcgccctccacatccctgattcctgttgttatggaaactgttgccagagatggaggttctctcggagtatctgggaactgtgcctttgagtttcctcaggctgctggaggaaaactgagactcagacaggaaagggaaggccccacagacaaggtagccctggccagaggcttgttttgtcttttggtttttatgaggtgggatatccctatgctgcctaggctgaccttgaactcctgggctcaagcagtctacccacctcagcctcctgtgtagctgggattatagattggagccaccatgcccagctcagagggttgttctcctagactgaccctgatcagtctaagatgggtggggacgtcctgccacctggggcagtcacctgcccagatcccagaaggacctcctgagcgatgactcaagtgtctcagtccacctgagctgccatccagggatgccatctgtgggcacgctgtgggcaggtgggagcttgattctcagcacttgggggatctgttgtgtacgtggagagggatgaggtgctgggagggatagaggggggctgcctggcccccagctgtgggtacagagaggtcaagcccaggaggactgccccgtgcagactggaggggacgctggtagagatggaggaggaggcaattgggatggcgctaggcatacaagtaggggttgtgggtgaccagttgcacttggcctctggattgtgggaattaaggaagtgactcatcctcttgaagatgctgaaacaggagagaaaggggatgtatccatgggggcagggcatgactttgtcccatttctaaaggcctcttccttgctgtgtcataccaggccgccccagcctctgagcccctgggactgctgcttcttaaccccagtaagccactgccacacgtctgaccctctccaccccatagtgaccggctgcttttccctaagccaagggcctcttgcggtcccttcttactcacacacaaaatgtacccagtattctaggtagtgccctattttacaattgtaaaactgaggcacgagcaaagtgaagacactggctcatattcctgcagcctggaggccgggtgctcagggctgacacgtccaccccagtgcacccactctgctttgactgagcagactggtgagcagactggtgggatctgtgcccagagatgggactgggagggcccacttcagggttctcctctcccctctaaggccgaagaagggtccttccctctccccaagacttggtgtcctttccctccacttcctcctgccacctgctgctgctgctgctgctaatcttcagggcactgctgctgcctttagtcgctgaggaaaaataaagacaaatgctgcgcccttccccag

SEQ ID NO: 4 - protein, 432 aa, homo sapiens
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 5 - DNA, 1851 bp, homo sapiens
  CDS (123)..(1523)
gggcgcgccagagacgcagccgcgctcccaccacccacacccaccgcgccctcgttcgcctcttctccgggagccagtccgcgccaccgccgccgcccaggccatcgccaccctccgcagccatgtccaccaggtccgtgtcctcgtcctcctaccgcaggatgttcggcggcccgggcaccgcgagccggccgagctccagccggagctacgtgactacgtccacccgcacctacagcctgggcagcgcgctgcgccccagcaccagccgcagcctctacgcctcgtccccgggcggcgtgtatgccacgcgctcctctgccgtgcgcctgcggagcagcgtgcccggggtgcggctcctgcaggactcggtggacttctcgctggccgacgccatcaacaccgagttcaagaacacccgcaccaacgagaaggtggagctgcaggagctgaatgaccgcttcgccaactacatcgacaaggtgcgcttcctggagcagcagaataagatcctgctggccgagctcgagcagctcaagggccaaggcaagtcgcgcctaggggacctctacgaggaggagatgcgggagctgcgccggcaggtggaccagctaaccaacgacaaagcccgcgtcgaggtggagcgcgacaacctggccgaggacatcatgcgcctccgggagaaattgcaggaggagatgcttcagagagaggaagccgaaaacaccctgcaatctttcagacaggatgttgacaatgcgtctctggcacgtcttgaccttgaacgcaaagtggaatctttgcaagaagagattgcctttttgaagaaactccacgaagaggaaatccaggagctgcaggctcagattcaggaacagcatgtccaaatcgatgtggatgtttccaagcctgacctcacggctgccctgcgtgacgtacgtcagcaatatgaaagtgtggctgccaagaacctgcaggaggcagaagaatggtacaaatccaagtttgctgacctctctgaggctgccaaccggaacaatgacgccctgcgccaggcaaagcaggagtccactgagtaccggagacaggtgcagtccctcacctgtgaagtggatgcccttaaaggaaccaatgagtccctggaacgccagatgcgtgaaatggaagagaactttgccgttgaagctgctaactaccaagacactattggccgcctgcaggatgagattcagaatatgaaggaggaaatggctcgtcaccttcgtgaataccaagacctgctcaatgttaagatggcccttgacattgagattgccacctacaggaagctgctggaaggcgaggagagcaggatttctctgcctcttccaaacttttcctccctgaacctgagggaaactaatctggattcactccctctggttgatacccactcaaaaaggacattcctgattaagacggttgaaactagagatggacaggttatcaacgaaacttctcagcatcacgatgaccttgaataaaaattgcacacactcagtggcaggcgatatattacccaggcaagaataaaaaagaaatcccatatcttaaagaaacagctttcaagtgcctttctgcagtttttcaggagcgcaagatagatttggaataggaataagctctagttcttaacaaccgacactcctacaagatttagaaaaaagtttacaacataatctagtttacagaaaaatcttgtgctagaatactttttaaaaggtattttgaataccattaaaactgcttttttttttccagcaagtatccaaccaacttggttctgcttcaataaatctttggaaaactcca

SEQ ID NO: 6 - protein, 466 aa, homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTFLIKTVETRDGQVINETSQHHDDLE

(19) United States
(12) Patent Application Publication
(10) Pub. No.: US 2005/0152995 A1
(43) Pub. Date: Jul. 14, 2005

(54) Methods and compositions for promoting axon regeneration and cell replacement therapy

(76) Inventors: Dong Feng Chen, Newton, MA (US); Kin-Sang Cho, Winchester, MA (US); Masumi Takeda, Boston, MA (US); Reiko Kinouchi, Hokkaido (JP)

Correspondence Address: FOLEY HOAG, LLP;PATENT GROUP, WORLD TRADE CENTER WEST, 155 SEAPORT BLVD, BOSTON, MA, 02110, US

(21) Appl. No.: 10/877,066

(22) Filed: Jun. 25, 2004

Related U.S. Application Data
(60) Provisional application No. 60/483,528, filed on Jun. 27, 2003.

(51) Int. Cl. A61K 33/00; A61K 31/19

(52) U.S. Cl. 424/722; 514/557

(57) ABSTRACT

Provided herein are methods and compositions for rendering a cellular environment permissive to axon regeneration and neural cell transplantation. Methods for stimulating axon regeneration in adult subjects are also disclosed. The methods may comprise contacting a tissue with an agent that prevents glial scar formation, such as by inhibiting reactive astroglial cells, and optionally an agent that increases bcl-2 protein levels in neural cells. Exemplary agents include astrotoxin for inhibiting reactive astroglial cells and lithium for increasing bcl-2 protein levels.

### CROSS-REFERENCE TO RELATED APPLICATIONS

This application claims the benefit of U.S. Provisional Application No. 60/483,528, filed Jun. 27, 2003, the contents of which are specifically incorporated by reference herein.

### STATEMENTS OF RIGHTS

This invention was made during the course of work supported by NIH grant EY012983. Thus, the U.S. Government may have certain rights in the invention.

### BACKGROUND

Neurons in the mammalian central nervous system (CNS), including the brain, spinal cord, and the retina, regenerate poorly after injury. The death of these cells, as a result of injury or disease, usually results in permanent functional loss or blindness. Although axons may regenerate early in mammalian development, later in development, axons in the mammalian central nervous system (CNS) lose their ability to regenerate after injury. The mechanisms of this growth failure are unclear. According to the prevailing view, CNS regenerative failure reflects both the intrinsic inability of adult CNS neurons to survive or reinitiate axonal growth and the lack of a permissive environment for such growth (1-4).

The adult CNS not only presents a non-permissive environment for the regeneration, the same mechanisms also prevent the successful use of neural transplantation to replace those cells lost due to disease or injury. Transplantation of neurons, even immature neurons and neural stem cells, into the mature CNS faces a major hurdle—the limited ability of these cells to survive, migrate, and establish morphological and functional connectivity with their hosts (1′-5′). This outcome is particularly prominent after retinal transplantation (2′). Even though some success has been achieved by transplanting stem cells (6′,7′), less than 1% of them repopulates and integrates into the normal adult retina (8′). Most grafted cells die, and the majority of those that survive do not migrate out of the injection site and lack axon- or dendrite-like processes that extend into the hosts (3′,7′,8′). Since immature neurons and neural stem cells, in general, are intrinsically capable of migrating, differentiating, and growing neurites, these findings imply that the normal retinal environment presents a barrier to graft integration and formation of synaptic function.

Thus, there is a need for methods that permit axon regeneration and neural cell transplantation.

### SUMMARY

The methods and compositions provided herein may promote a permissive environment for axon regeneration or cell replacement, such as replacing neural cells, by preventing or reducing glial scar formation by, e.g., inhibiting the formation or function of reactive astroglial cells, such as astrocytes and Mueller cells. The activity of reactive astroglial cells may be inhibited by contacting astroglial cells with an axon regeneration-promoting amount of astrotoxin or analog thereof. Further, the activity of reactive astroglial cells may be inhibited by suppressing the expression of glial fibrillary acid protein (GFAP) and vimentin (Vim) in astroglial cells.

In addition to inhibiting reactive astroglial cells, promoting mammalian axon regeneration may involve increasing bcl-2 activity or protein level. Bcl-2 activity or protein level may be increased by contacting the cell or axon with lithium or analog thereof. Promoting mammalian axon regeneration may also involve simultaneous or sequential contact of the axon or cell with one or more neuron stimulating factors, such as fibroblast growth factor, ciliary neurotrophic factor, nerve growth factor or brain-derived neurotrophic factor from about 0.01 to about 10,000 mg/kg body weight of the subject.

The methods and compositions provided herein may be useful for introducing a cell into a subject by inhibiting reactive astroglial cells in the vicinity of the location of which the cell will be introduced and introducing a cell, such as a neural cell, a neural progenitor cell, or a stem cell, into the subject. The method may involve inhibiting astroglial cells by contacting the astroglial cells with a regenerating-promoting amount of astrotoxin or analog thereof. Alternatively, the method may involve inhibiting astrocytes by suppressing the expression of GFAP or Vim. Further, the method may be used to increase the activity or protein levels of bcl-2, by promoting the transcription of the endogenous bcl-2 or by introducing an exogenous bcl-2 gene into the cell.

The methods and compositions provided herein may create a permissive environment for axon regeneration or cell replacement, such as replacing a neural cell, in a subject that would benefit from axon regeneration or cell replacement. In creating a permissive environment for axon regeneration or cell replacement, the method may involve administering to the subject a pharmaceutically effective amount of an agent that inhibits reactive astroglial cells at the site where axon regeneration or cell replacement is desired. Inhibiting reactive astroglial cells may be achieved by administering astrotoxin or an analog thereof from about 0.01 to about 10,000 mg/kg body weight of the subject. Alternatively, the agent that inhibits reactive astroglial cells may suppress the expression of GFAP or Vim. The method may promote axon regeneration or cell replacement in a mammal, such as a human, in the peripheral nervous system, central nervous system or ocular tissue, e.g., retina.

The methods and compositions provided herein may be used in an assay to identify an agent that promotes a permissive environment for axon regeneration or cell transplantation by, for example, contacting a reactive astroglial cell with a test agent and determining the effect of the test agent on the activity of the astroglial cell. A lower activity of the astroglial cells in the presence of the test agent indicates that the agent promotes a permissive environment for axon regeneration or cell transplantation. The assay may determine the effect of the agent on the activity or protein level of GFAP or Vim or other proteins specifically expressed in activated glial cells. Another assay comprises contacting an astrocyte with a test agent and determining the effect of the test agent on the activity of the astroglial cells. Decreasing the number of astroglial cells or preventing astroglial cell hypertrophy in the presence of the test agent indicates that the agent promotes a permissive environment for axon regeneration or cell transplantation. Further, the assay may determine the effect of the agent in killing astroglial cells.

### BRIEF DESCRIPTION OF THE FIGURES

FIG. 1 shows a robust and rapid optic nerve regeneration in P3 Bcl-2tg mice. (A-F) Photomicrograph montages of adjacent longitudinal optic nerve sections from wild-type (A, C, E) and Bcl-2tg (B, D, F) mice 24 h after optic nerve crush, showing the morphologies of regenerating axons. The sections were stained with cresyl violet (A and B) or anti-GAP-43 (E and F). Insets (C and D) show higher-power views of axon morphology (100×). Asterisk indicates the crush site. Scale bar: 250 mm; 25 μm (inset). (G-J) High-magnification (40×) and confocal (100×) epifluorescence photomicrograph montages of adjacent longitudinal optic nerve sections stained with anti-NF-M, showing axon morphologies in wild-type (G) and Bcl-2tg (H-J) mice on day 2 after injury. (I and J) Insets in (H) show confocal images of growth cones (arrowheads). Asterisks indicate the crush site. Scale bars: 100 μm; 5 μm (I and J).

FIG. 2 shows that a majority of RGC axons in Bcl-2tg mice regenerate and reach the ipsilateral brain targets within 4 days. (A-F) Epifluorescence photomicrographs of coronal brain sections from a Bcl-2tg mouse examined on day 4. Note green fluorescence (CTB-F labeling) in the ipsilateral SC and pretectal nuclei (PT) (A), dorsal (dLGN) and ventral LGN (vLG) (B), and the optic tract (C). Weak fluorescence is present in the corresponding contralateral targets (D-F). Dotted lines outline the SC and dLGN. Arrows indicate positive fluorescence in the optic tract. Scale bar: 200 mm. (G-L) Quantitative assessment of axon regeneration. Photomicrographs of FluoroGold-labeled RGCs in whole-mount retinas from injured and uninjured wild-type and Bcl-2 tg mice on day 11 after optic nerve crush. Scale bar: 50 μm. (K and L) Bar charts showing the number of retrogradely labeled RGCs and the traveling distance of regenerating axons on days 1-4. Values are mean±S.E.M.

FIG. 3 shows that the onset of optic nerve regenerative failure in P5 Bcl-2tg mice coincides with astrocyte maturation. (A and B) Quantification of retinal axon regrowth in retina-midbrain slice co-cultures. Values are mean±S.D. (**P<0.01; ***P<0.001). (C-F) Photomicrograph montages of adjacent longitudinal optic nerve sections at day 4 after optic nerve crush in a P5 wild-type mouse (C and E) and a Bcl-2tg mouse (D and F). Asterisk indicates the crush site. Scale bar: 250 μm. (G and H) Western blot analysis (G) and RT-PCR (H) reveal developmental expression patterns of myelin/oligodendrocyte-associated proteins and astrocyte markers in E14-P14 mouse midbrains. (I) Western blot analysis of GFAP expression in normal P2 and P7 midbrain tissues and those injured 2 days earlier. (J) Western blot analysis confirms the absence of myelin proteins, MBP, and MAG in the midbrains of jimpy mice. (K) Quantification of retinal axon regrowth into midbrain slices of P14 wild-type and jimpy mice in retina-midbrain slice co-cultures. Values are mean±S.E.M. (P=0.6).

FIG. 4 shows robust optic nerve regeneration in adult Bcl-2tg mice after treatment with astrotoxin. (A-H) Photomicrograph montages of adjacent longitudinal optic nerve sections on day 8 after optic nerve crush in adult wild-type (A, C, E, G) and Bcl-2tg (B, D, F, H) mice. Asterisk indicates the crush site. Scale bar: 250 μm. (I and J) Electron micrographs of optic nerve sections taken from wild-type (I) and Bcl-2tg (J) mice treated with astrotoxin. The cross sections of the optic nerve were collected at 0.5 mm posterior to the crush. Asterisks indicate regenerating axons. Scale bar: 1 μm. (K) Quantification of regenerating axons from optic nerve sections. Values are mean±S.E.M. (***P<0.001).

FIG. 5 shows robust optic nerve regeneration and innervation of brain targets in postnatal Bcl-2tgGFAP−/−Vim−/− mice. (A-H) Photomicrograph montages of adjacent longitudinal optic nerve sections on day 4 after optic nerve crush in P14 GFAP−/−Vim−/− (A, C, E) and Bcl-2tgGFAP−/−Vim−/− (B, D, F) mice. Asterisk indicates the crush site. Scale bar: 250 μm; 50 μm in (E) and 5 μm in (F). (G-I) Photomicrographs of whole-mount retinas from Bcl-2tg (G), GFAP−/−Vim−/− (H), and Bcl-2tgGFAP−/−Vim−/− (I) mice on day 11 after optic nerve injury, showing FluoroGold-labeled RGCs. Scale bar: 50 μm. (J) Quantification of the number of retrogradely labeled RGCs. Values are mean±S.E.M. (***P<0.001).

FIG. 6 shows failure of graft integration and induction of reactive gliosis after retinal transplantation in wild-type mice. A, B, Retinal grafts taken from P0 EGFP transgenic mice and transplanted into the subretinal space (A) or vitreous cavity (B) of adult wild-type mice show no signs of neural migration, neurite outgrowth, or integration into the host retina. Scale bar, 1 mm. c-f, Imunofluorescence staining (red) of normal retinal sections (C, D) and sections from the retinas with implants (E, F) with antibodies against the glial markers GFAP (C, E) and vimentin (D, F). Note increased expression of GFAP and vimentin in the transplanted retina at the injection site (*) and at the interface between the graft and the host retina (arrowheads). Scale bar, 200 μm. g, A representative western blot of triplicate experiments using retinal proteins before and after the transplantation, probed with antibody against chondroitin sulfate proteoglycan (CS-56). Beta-tubulin was used as loading control.

FIG. 7 shows robust neural graft integration into the retina of adult GFAP−/−Vim−/− mice. A, B, Merged fluorescence and phase-contrast images of retinal sections reveal that transplanted EGFP-positive cells (green) aggregated around the injection site in the subretinal space of wild-type host (WILD-TYPE ) (A). Transplanted cells migrated widely in the retina (R) of GFAP−/−Vim−/− mouse (GV) (B) and localized primarily in the host GCL. Arrows point to EGFP-positive cells. Scale bar, 500 μm. C, Photomicrograph of an optic nerve section shows EGFP-positive neurites extending into the optic nerve of a GFAP−/−Vim−/− mouse. Scale bar, 20 μm. D, E, Flourescence images of grafted EGFP-positive cells in retinal whole-mount preparations. EGFP-positive cells grafted to the GFAP−/−Vim−/− mouse (E) grew extensive neurites into the host retina (background) while those in the wild-type host (D) rarely grew neurites. Scale bar, 50 μm. F-H, The numbers of grafted cells that repopulated (F, G) and the percentages of cells that regenerated neurites longer than 1 (>l×) or 3 (>3×) cell body lengths (H) into the host retina, analyzed in retinal whole-mounts (F, H) and retinal sections (G) of wild-type and GFAP−/−Vim−/− mice. Data represent mean±S.D. ***P <0.001 by two-tailed t test.

FIG. 8 shows morphological integration of EGFP neurons into the GCL of GFAP−/−Vim−/− mice. A-F, Confocal images of EGFP-positive cells in retinal sections (A, B, D-F) and whole-mount preparation (C) of wild-type (A, D) and GFAP−/−Vim−/− (B, C, E, F) mice at 3-21 days post-injections. A-C, Images obtained at 21 days show the simple neurite morphology of a transplanted cell in the wild-type host (WILD-TYPE ) (A) and extensive morphological integration of grafted cells into the retinas of GFAP−/−Vim−/− hosts (GV) (B, C). Transplanted cells in a GFAP−/−Vim−/− mouse extended a single axon-like process parallel to the retinal surface (arrowhead) and branched dendritic tree structures (arrow) into the host retina. D-F, Images of retinal sections from wild-type (D) and GFAP−/−Vim−/− (E, F) mice 3 (E), 7 (D), and 14 (F) days after transplantation. Note the robust cell migration from the subretinal space (SUB) into the GCL in GFAP−/−Vim−/− mice but not in wild-type mice. Arrows point to the migrating neurons. Scale bars in (A-C), 5 μm and (D-F), 20 μm. G, Quantification of grafted cell repopulation in different layers of the host retina 21 days after transplantation. Data represent mean±S.D. ***P<0.001, by two-tailed t test. IPL, inner plexiform layer; ONL, outer nuclear layer; INL, inner nuclear layer.

FIG. 9 shows morphological integration of transplanted cells into the retinas of adult GFAP−/−Vim−/− mice. Confocal images of immunofluorescence labeled retinal sections from GFAP−/−Vim−/− mice 21 days after transplantation. C, F, I, L, O, Overlay images of EGFP-positive cells (A, D, G, J, M) transplanted into the retinas of a GFAP−/−Vim−/− hosts and stained with primary antibodies against NF-L (B, N), MAP2 (E), Thy1.2 (H), or Bm-3b (K). Labeling of anti-Thy1.2 shows the inner plexiform layer (INL) of the host retina (H), and the overlay image (I) indicates that EGFP-labeled cells extend dendritic structures (arrowhead) into the IPL and a single axon-like process (arrow) running parallel into the nerve fiber layer of the host retina. Note labeling of host RGCs by anti-Bm-3b (arrowheads) in (K, L). IPL: inner plexiform layer. M-O, Orthogonal projections of confocal images showing again that EGFP cells are positively labeled by antibody against NF-L. Scale bars, 20 μm.

FIG. 10 shows neuronal repopulation and integration in the retinas of wild-type (WILD-TYPE ), GFAP−/−Vim−/− (GV), GFAP−/−G), and Vim−/− (V) mice. A-D, Morphologies of repopulated EGFP-positive cells in the retinal whole-mounts (background) of wild-type (A), GFAP−/− (B), Vim−/− (C), and GFAP−/−Vim−/− (D) mice at 10 days after transplantation. Scale bar, 100 μm. E-F, Quantification, in retinal whole-mounts, of grafted cells that repopulated (E) or extended neurites longer than 1 (>1×) or 3 (>3×) cell body lengths (F) into the host retinas of wild-type, GFAP−/−, Vim−/−, and GFAP−/−Vim−/− mice. Data represent mean±S.D. *P<0.05 and ***P<0.001 by ANOVA.

FIG. 11 shows morphology of reactive astrocytes, Müller glial cells, and the ILM in the retinas of GFAP−/−Vim−/− mice. A-D, Electron photomicrographs showing cellular processes (white arrows) of reactive astrocytes (A, B) and Müller cells (C, D)(black arrows) in the retinal sections of wild-type (A, C) and GFAP−/−Vim−/− (B, D) mice. Scale bars, 10 μm. E-H, Photomicrographs of retinal sections from wild-type (E, F) and GFAP−/−Vim−/− (G, H) mice after removal of the lens. The sections were stained with hematoxylin and eosin (E, G) or anti-alpha-laminin antibodies (F, H) and show the relative position of the ILM (arrowheads) within the retina. Scale bar, 200 μm. * indicates the space between the retina and ILM.

FIG. 12 shows that the intake of lithium-containing diet increases lithium concentration in serum and upregulates Bcl-2 expression. (A) Graph showing a significant increased in lithium concentration in lithium-treated mice than the control wild-type mice consumed control diet (Student's t-test, p<0.00001). Values are mean±S.D. (B) RT-PCR results shows the induction of Bcl-2 mRNA in the retinas of lithium-treated mice. The Bcl-2 mRNA expressed in the retinas derived from lithium-treated wild-type mice and the positive control Bcl-2 transgenic (tg) mice but not in the wild-type mice that consumed a control diet. The expression of a house-keeping gene, G3PDH, served as an internal control.

FIG. 13 shows that localization of lithium induced Bcl-2 expression in adult mouse retina. Epifluorescence photomicrographs of retinal sections derived from mice fed with a control diet (A-C) or a lithium-containing diet (D-F). DAPI staining localizes the nuclear layers in retina (B, E). The arrow marks the co-localization of Bcl-2 protein in the ganglion cell layer (GCL). The insert is an enlarged image of the cells indicated by the arrrow (F). There is undetectable signal of Bcl-2 protein in the control retina (A-C). Scale=10 μm.

FIG. 14 shows that simultaneous administration of lithium and astrotoxin promotes robust regeneration of the severed optic nerves in adult wild-type mice. Montages of photomicrographs showing optic nerve sections in adult wild-type mice 8 days after optic nerve crush (A-L). First row: Cresyl violet staining identifies the crushed site of the optic nerve, which is marked by an asterisk. Second row: GAP-43 staining reveals the length of regenerating axons along the crushed optic nerve. Numerous GAP-43 positive axons regenerate posterior to the crush site in the mice receiving lithium and astrotoxin simultaneously (J) but no regenerating axons were observed posterior to the crushed site in the other groups (B and F). Third row: Strong GFAP staining extends along the crushed optic nerve. In the groups of mice receiving astrotoxin, there is a track of GFAP-negative area along the optic nerve (G and K). In the group of mice receiving lithium and astrotoxin simultaneously, the overlay shows that the regenerating axons fill the GFAP-negative area of the crushed optic nerve,which is astrocyte-ablated (L). Scale=250 μm.

FIG. 15 shows the growth cone-like structure of regenerating axons in the crushed optic nerve. In the mice that received lithium-containing diet and astrotoxin simultaneously, many GAP-43 labeled regenerating axons with a growth cone-like structure at their expanding tip are visible (arrow marked). Scale=5 μm.

FIG. 16 shows a graph indicating the length of the regenerating axons in the crushed optic nerve. The length of regenerating axons in the mice simultaneously receiving lithium (Li) and astrotoxin (AA) is significantly longer than that in the other groups (p<0.001). Values are mean±S.E.M.

FIG. 17 shows that a lithium-containing diet exerts no effect on prevention of RGCs death. (A) The graph shows the density of surviving RGCs, which are FluoroGold (FG) pre-labeled, in normal retinas (N) and retinas with optic nerve lesion (L) in the wild-type mice. There is no significant difference between the groups of lithium-treated or non-lithium treated mice (P>0.05). Photomicrographs showing the surviving FG-labeled RGCs in normal retina (B) and lesioned retina (C) derived from lithium-treated mice. Arrow marks the microglia with a feature of elongated cell body and fine processes. Scale=50 μm.

### DETAILED DESCRIPTION

Definitions

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

The terms “astrocytes” and “astroglial cells” refer to a type of glial cells that become reactive and upregulate intermediate filament (IF) proteins, such as glial fibrillary acid protein (GFAP) and vimentin (Vim), under pathological conditions or after transplantation in the brain and retina. “Astroglial cells” include both Muller glial cells (in the retina) and astrocytes. Astrocytes are star-shaped cells whose processes extend into the surrounding neuropil, and they are extensively coupled in a network—the astrocyte syncytium. There ar two types of astrocytes, the fibrous and protoplasmatic types, respectively, that are divided according to morphology and their location in white or gray matter. Fibrous astrocytes are predominantly found in the myelinated areas and have a star-like morphology with thin, usually unbranched, processes spreading out symmetrically from the cell body. The processes, rich in intermediate filaments, extend over long distances and frequently form the end-feet on blood vessels. Protoplasmatic astrocytes have shorter and highly branched processes of varying dimensions that ensheathe neuronal cell bodies and their processes. They form the end-feet on blood vessels and they also make contact with the pial surface.

The phrase “astrotoxin or analog” refers to the class of compounds of formula I and salts thereof.

As used herein, the phrase “axonal growth” or “axon growth” refers to the elongation or extension of an axon of a neural cell. An axon can elongate for distances of microns to meters. Extension or elongation of an axon is also referred to as “regeneration” of the axon of a neural cell. In one embodiment, axon regeneration results in the reestablishment of nerve cell connectivity.

The phrase “bcl family member” and “bcl polypeptide” include polypeptides, such as bcl-2 and other members of the bcl family. Bcl family member is meant to include within its scope fragments of a bcl family member which possess a bcl bioactivity. In other embodiments “bcl family members” include polypeptides which comprise bcl domains that confer bcl bioactivity, such as, for example, BH1, BH2, or BH4. Exemplary bcl family members include: bcl-2, Bcl-xL, Bcl-xs, Bad, Bax, and others (Merry, D. E. et al. Development 120:301 (1994); Nifiez, G. et al. Immunol. Today 15, 582-588 (1994)). Human bcl-xL nucleotide and amino acid sequences can be found, e.g., as GenBank no. Z23115, described in Boise et al. (1993) Cell 74:597. Human bcl-2 nucleotide and amino acid sequences can be found, e.g., as GenBank no. M14745, described in Cleary et al. (1986) Cell 47:19. Agents that “modulate” the expression or bioactivity of a bcl family member is meant to include agents which either up or downregulate the expression or bioactivity of a bcl family member. In preferred embodiments, a modulating agent upregulates the expression or bioactivity of a bcl family member. Agents which upregulate expression make a quantitative change in the amount of a bcl family member in a cell, while agents which upregulate the bioactivity of a bcl family member make a qualitative change in the ability of a bcl family member to perform a bcl bioactivity. Such agents can be useful therapeutically to promote axonal growth in a cell. Accordingly, the subject methods can be carried out with BCL family member modulating agents, e.g., those described herein, such as, nucleic acids, peptides, and peptidomimetics, or modulating agents identified in drug screens which have a bcl family member bioactivity, for example, which agonize or antagonize the effects of a BCL family member protein. In one aspect, bcl modulating agents are nucleic acids encoding a bcl family member polypeptide which are introduced into a cell. Exemplary agents are bcl family member nucleic acids, for example in plasmids or viral vectors.

“Cell replacement” refers to cell, tissue or organ transplantation.

“Diminished,” as applied to axonal growth, as used herein is meant to include states in which axonal growth is absent as well as those in which it is reduced, e.g., by about 10%, 30%, 50%, 75%, 90% or 95%.

“GFAP” refers to “glial fibrillary acid protein” which is one of the major intermediate filament proteins of mature astrocytes. It is used as a marker to distinguish astrocytes from other glial cells during development. Mutations in this gene cause Alexander disease, a rare disorder of astrocytes in the central nervous system. The nucleotide and amino acid sequences of human GFAP are set forth as SEQ ID NOs: 3 and 4, respectively, and correspond to GenBank Accession Nos. NM_{—}002055 and NP_{—}002046, respectively.

“Inhibiting the expression” and “suppressing the expression” of a protein refer to decreasing the level of the protein by at least 10%, 30%, 50%, 80%, 90%, 95% or 100%.

The term “nucleic acid” refers to polynucleotides such as deoxyribonueleic acid (DNA), and, where appropriate, ribonucleic acid (RNA). The term should also he understood to include, as equivalents, analogs of either RNA or DNA made from nucleotide analogs, and, as applicable to the embodiment being described, single (sense or antisense) and double-stranded polynucleotides.

“Neuron,” “neuronal cell” and “neural cell” are used interchangeably to refer to nerve cells, i.e., cells that are responsible for conducting nerve impulses from one part of the body to another. Most neurons consist of three distinct portions: a cell body, soma or perikaryon, which contains a nucleus and two kinds of cytoplasmic processes: dendrites and axons. Dendrites are usually highly branched, thick extensions of the cytoplasm of the cell body. An axon is usually a single long, thin process that is highly specialized and conducts nerve impulses away from the cell body to another neuron or muscular or glandular tissue. Along the length of an axon, there may be side branches called “axon collaterals.” Axon collaterals and axons may terminate by branching into many fine filaments called “axon terminals.” The distal ends of axon terminals are called “synaptic end bulbs,” which contain synaptic vesicles that store neurotransmitters. Axons may be surrounded by a multilayered, white, phospholipid, segmented covering called the myelin sheath. Axons containing such a covering are “myelinated.” Neurons include sensory neurons, which transmit impulses from receptors in the skin, sense organs, muscles, joints, and viscera to the brain and spinal cord and from lower to higher centers of the CNS. A neuron can also be a motor (efferent) neuron convey impulses from the brain and spinal cord to effectors, which may be either muscles or glands, and from higher to lower centers of the CNS. Other neurons are association (connecting or interneuron) neurons which carry impulses from sensory neurons to motor neurons and are located in the brain and spinal cord. Examples of association neurons include stellate cells, cells of Martinotti, horizontal cells of Cajal, pyramidal cells, granule cells and Purkinje cells. The processes of afferent and efferent neurons arranged into bundles are called “nerves” when located outside the CNS or fiber tracts if inside the CNS. A neural cells include neural progenitor cells and neural stem cells.

“Neural tissue” includes any tissue that comprises a neural cell or a nerve, e.g., peripheral nerves, ganglia, cranial nerves, the spinal cord surface, deep spinal cord tissue, deep brain tissue, brain surface tissue, optic nerve, and retina.

“Ocular tissue” includes nerves or nerve tissue in, or relating to, the eye, e.g., the optic nerve. Ocular tissue also includes the retina.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interference RNA (siRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a nonnatural arrangement.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrasternal injection and infusion.

A “patient”, “subject” or “host” refers to either a human or a non-human animal.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The term phrase “permissive environment” refers to an environment that is favorable for axon growtwth and/or neural cell transplantation. A permissive environment can be created by modifying an area near a regenerating axon or along the path that a regenerating axon navigates to reach its target cells to reduce or eliminate aspects of the area that impede axon regeneration. Further, “creating a permissive environment” also refers to modifying an area near a cell transplanted into the area to reduce or eliminate aspects of the area that impede the survival, migration, neurite extension or connection with other neurons of the transplanted cell. The “permissive environment” may be in vivo, in vitro or ex vivo.

The term “prophylactic” or “therapeutic” treatment is art-recognized and refers to administration of a drug to a host. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The term “pharmaceutically-acceptable salts” is art-recognized and refers to the relatively non-toxic, inorganic and organic acid addition salts of compounds, including, for example, those contained in compositions described herein.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “protein” (when consisting of a single polypeptide chain), “polypeptide”, and “peptide” are used interchangeably herein.

The phrase “subject” as used herein is meant to encompass mammals. As such the methods and compositions presented herein is useful for the treatment of domesticated animals, livestock, zoo animals, etc. Examples of subjects include humans, cows, cats, dogs, goats, and mice.

As used herein, the term “state characterized by diminished potential for axonal growth or regeneration, or cell replacement” is meant to encompass a state or disorder which would benefit from the simulation of axonal growth or regeneration, or cell replacement.

The terms “systemic administration,” “administered systemically,” “peripheral administration” and “administered peripherally” are art-recognized and refer to the administration of a subject composition, therapeutic or other material other than directly into the central nervous system, such that it enters the patient's system and, thus, is subject to metabolism and other like processes.

“Transcriptional regulatory sequence” is a generic term used throughout the specification to refer to DNA sequences, such as initiation signals, enhancers, and promoters, which induce or control transcription of protein coding sequences with which they are operable linked. In preferred embodiments, transcription of one of the recombinant genes is under the control of a promoter sequence (or other transcriptional regulatory sequence) which controls the expression of the recombinant gene in a cell-type which expression is intended. It will also be understood that the recombinant gene can be under the control of transcriptional regulatory sequences which are the same or which are different from those sequences which control transcription of the naturally-occurring forms of genes as described herein.

“Treating” a condition or disease refers to curing as well as ameliorating at least one symptom of the condition or disease or preventing the disease from worsening.

A “vector” is a self-replicating nucleic acid molecule that transfers an inserted nucleic acid molecule into and/or between host cells. The term includes vectors that function primarily for insertion of a nucleic acid molecule into a cell, replication of vectors that function primarily for the replication of nucleic acid, and expression vectors that function for transcription and/or translation of the DNA or RNA. Also included are vectors that provide more than one of the above functions. As used herein, “expression vectors” are defined as polynucleotides which, when introduced into an appropriate host cell, can be transcribed and translated into a polypeptide(s). An “expression system” usually connotes a suitable host cell comprised of an expression vector that can function to yield a desired expression product.

“Vimentin” is an intermediary filament. The nucleotide and amino acid sequences of human vimentin are set forth as SEQ ID NOs: 5 and 6, respectively, and correspond to GenBank Accession Nos. NM_{—}003380 and NP_{—}003371, respectively.

Exemplary Methods and Compositions

Provided herein are methods and compositions for promoting a permissive environment for axonal regeneration or cell replacement of neurons (e.g., transplantation of neurons). Such permissive environment may be created by inhibiting the formation or function of reactive astroglial cells. Reactive astroglial cells can be inhibited by either deactivating reactive astroglial cells or by preventing astroglial cells from being activated, such as by preventing astroglial cells from proliferating, becoming hypertrophic, from scarring, or from producing molecules characteristic of reactive astrocytes, e.g., GAFP, vimentin, apolipoprotein D, myocilin, and various chondroitin sulfate proteoglycans (CSPGs) that include hyalectans, neurocan, versican, brevican, NG2, phosphacan. Reactive astroglial cells can also be inhibited by, e.g., contacting the cells with an interleukin, such as IL-10, or with macrophage inhibitory factor (Balasingam et al. (1996) J. Neuroscience 16:2945). “Inhibiting reactive astroglial cells” refers to inhibiting at least some of the reactive astroglial cells in a population of reactive astroglial cells, e.g., at least about 10%, 30%, 50%, 75%, 90% or 95%. Preferably, the inhibition is sufficient to result in a permissive environment for axon regeneration or cell transplantation. Inhibiting astroglial cells astrocytes can be accomplished by, for example, contacting astroglial cells with a “regenerating-promoting amount” of astrotoxin (L-alpha-aminoadipate) or an analog thereof, i.e., an amount sufficient for permitting at least some axonal regeneration. A permissive environment may also be created by inhibiting reactive astroglial cells by reducing the activity or protein levels, e.g., by suppressing the expression, of intermediate filament proteins, such as glial fibrillary acid protein (GFAP) or vimentin (Vim), or other astrocyte-associated proteins, such as apolipoprotein D, myocilin, or CSPGs, in astroglial cells. A permissive environment may also be created by killing astroglial cells.

The reactivity of astroglial cells can be determined, e.g., histochemically or biochemically, such as by measuring the level of expression of GFAP, apolipoprotein D, myocilin, CSPGs, electrophysiologically, such as by measuring the reduction of inwardly rectifying K+ channel currents; or morphologically, such as by measuring the formation of astroglial scar tissue.

Also provided herein are methods and compositions for promoting axonal regeneration or cell replacement of, e.g., neurons. In one embodiment the methods and compositions herein involve (i) inhibiting astroglial cells, such as by contacting astroglial cells with a regenerating-promoting amount of astrotoxin or an analog thereof, or by suppressing the expression of intermediate filament proteins, such as glial fibrillary acid protein (GFAP) or vimentin (Vim) or other reactive astroglial cells-associated proteins, such as apolipoprotein D, myocilin, or CSPGs in astroglial cells; and (ii) stimulating axon regeneration, e.g., by increasing the activity or level of a bcl-2 protein. For example, the activity or level of a bcl-2 protein may be induced by contacting the axon or cell with lithium or an analog thereof.

Provided herein are also methods and compositions for creating a permissive environment or promoting axon regeneration or cell replacement of, e.g., neurons, in a subject having diminished potential for axonal growth or regeneration, or cell replacement. In one embodiment, the method comprises administering to a subject in need thereof a pharmaceutically effective amount of astrotoxin or analog thereof. The method may also involve inhibiting the expression of intermediate filament proteins, such as GFAP and Vim, or other reactive astrocyte-associated proteins, such as apolipoprotein D, myocilin, and CSPGs. Methods for promoting axon regeneration or cell replacement may involve increasing the activity or level of a bcl-2 protein. In another embodiment, the activity or level of a bcl-2 protein is induced by contacting the axon or cell with lithium or an analog thereof.

A method for promoting axon regeneration at a site of neural injury may comprise administering at the site of injury or in the vicinity of it (i) a therapeutically efficient amount of an agent that induces a permissive environment; and (ii) an agent that increases axon regeneration. The agents may be administered simultaneously or consecutively. For example, a subject having a spinal cord injury may be treated by administration at the site or in the vicinity of the site of injury of a therapeutically efficient amount of astrotoxin or analog thereof and a therapeutically efficient amount of a salt of lithium. For example, therapeutically efficient amounts of astrotoxin that are delivered to the site of injury or the vicinity of it range from about 0.1 mg/ml, 1 mg/ml, 10 mg/ml or 100 mg/ml solutions. A salt of lithium may be administered orally, e.g., in doses similar to those that are currently used for treating other conditions with lithium.

A method for administering neural cells to a subject having a neural injury may comprise administering at the site of injury or in the vicinity of it (i) a therapeutically efficient amount of an agent that induces a permissive environment; and (ii) neural cells. Optionally the method includes administration of an agent that promotes axonal regeneration.

Further provided are pharmaceutical compositions comprising an agent that creates an environment favorable to axonal growth thereof in a pharmaceutically acceptable excipient. Exemplary compositions include an agent that inhibits reactive astroglial cells, e.g., astrotoxin or an analog thereof, and agents that reduce GFAP, Vim, apolipoprotein D, myocilin or CSPGs activity or protein level. The composition may also comprise an agent that promotes axon regeneration, such as lithium or a salt or analog thereof.

Astrotoxin analogs include compounds having formula 1: 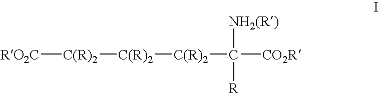
wherein, independently for each occurrence,
R represents H, alkyl, or aryl; and
R′ is absent or represents H.

Also included are pharmaceutically acceptable addition salts of astrotoxin or analogs of formula I. In cases wherein the astrotoxin or analogs of formula I may have one or more chiral centers, unless specified, the astrotoxin or analog thereof comprises each unique racemic compound, as well as each unique nonracemic compound.

Also included are prodrugs of the astrotoxin or analogs of formula I. Prodrugs are considered to be any covalently bonded carriers which release the active parent drug in vivo.

In a further embodiment, an astrotoxin analog comprises the formula I and the attendant definitions, wherein at least one R is alkyl. In a further embodiment, at least one R is methyl. In a further embodiment, astrotoxn is α-aminoadipate (25, 26). In a further embodiment, the analog is α-aminoadipic acid. In a further embodiment, the analog is (2S,4S)-4-methyl-aminoadipate. In a further embodiment, the analog is (2S,5S)-5-methyl-aminoadipate. In a further embodiment, the analog is (2S,5R)-5-methyl-aminoadipate (Guldbrandt, et al. Chirality 14: 351-63 (2002)). Astrotoxin has been shown to selectively kills astrocytes and has a minimal effect on surrounding neurons or myelin.

Methods for increasing the activity or level of a bcl-2 protein in a cell may comprise contacting the cell with a compound that increases bcl-2 protein levels or activity. Compounds that increase bcl-2 protein levels include those that stimulate transcription of the gene encoding bcl-2. Bcl-2 protein levels may also be increased by introducing into the cell a nucleic acid that encodes a bcl-2 protein or biologically active portion thereof. The nucleic acid may be operably linked to a transcriptional control element. An exemplary nucleic acid comprises or consists of at least a portion of the nucleic acid encoding human bcl-2 having the nucleotide sequence set forth as SEQ ID NO: 1 (GenBank Accession No. M14745 encoding a protein having GenBank Accession No. AAA35591). Other nucleic acids encoding a human bcl-2 protein, comprising, e.g., SEQ ID NO: 2 or a portion thereof, may also be used. Furthermore, nucleic acids that hybridize to SEQ ID NO: 1 under stringent hybridization conditions, e.g., a hybridization step and/or wash step in 0.2×SSC at 65° C., can also be used. Nucleic acids that encode a protein having an amino acid sequence that is at least about 90%, 95%, 98% or 99% identical to SEQ ID NO: 2 may also be used. Nucleic acids can be part of a vector and may be introduced into cells according to methods known in the art. Exemplary methods comprising the use of liposomes or viral vectors are further described herein. In yet another embodiment, a bcl-2 protein or biologically active portion thereof is introduced into a cell according to methods known in the art.

Also provided herein are methods for identifying a compound that (1) creates a permissive environment for axonal regeneration or cell replacement of, e.g., neurons, such as neural progenitor cells and neural stem cells, or (2) promotes axonal regeneration or cell replacement of, e.g., neurons, neural progenitor cells or neural stem cells. The method comprises, e.g., contacting the axon, neuron, neural progenitor cell, or neural stem cell with a test agent and determining the effect of the test agent on inhibiting reactive astroglial cells and/or promoting axon regeneration or cell replacement of, e.g., neurons, neural progenitor cells, or neural stem cells. The method may comprise contacting the axon, neuron, neural progenitor cell, or neural stem cell which may be in a composition or tissue comprising other cells, such as astroglial cells, with a test agent and determining the effect of the agent in suppressing GFAP and Vim expression and/or promoting axon regeneration. One exemplary method comprises contacting a tissue sample comprising astroglial cells with a test compound and a cell, e.g., a neuron, neural progenitor cell or neural stem cell, and determining the effect of the compound on the implantation of the cell into the tissue. The tissue sample may be further contacted with lithium or salt thereof. Alternatively, a tissue or cells comprising neurons having severed axons and astroglial cells may be contacted with a test compound in the presence of an agent that promotes axon regeneration, e.g., lithium, and the effect of the test compound on axon regeneration is determined. The presence of axon regeneration in a tissue or cells that was contacted with a test compound relative to one that was not contacted with the compound indicates that the test compound is an agent that renders the environment permissive. The test agent and cell can be contacted simultaneously or successively with the tissue sample. The effect of the test agent may be compared to that of astrotoxin, i.e., astrotoxin can serve as a positive control.

Another exemplary method comprises (i) providing a tissue or cells comprising astroglial cells and neurons having severed axons; (ii) contacting the tissue or cells with a test agent and an agent that renders the environment permissive, e.g., astrotoxin; and (iii) determining the effect of the test compound on axon regeneration. The presence of axon regeneration in the presence of the test compound relative to the absence of the test compound indicates that the test compound induces axon regeneration.

Provided herein are methods and compositions for treating subjects having a state characterized by diminished potential for axonal growth or regeneration, or cell replacement of neurons. Such a state may occur normally, as in adult neurons of the CNS, or because of a pathologic condition. Exemplary states “characterized by diminished potential for axonal growth or regeneration, or cell replacement of neurons” include neurological conditions derived from injuries of the spinal cord or compression of the spinal cord, or complete or partial transection of the spinal cord. For example, injuries may be caused by: (i) acute, subacute, or chronic injury to the nervous system, including traumatic injury (e.g. severing or crushing of a neuron(s)), such as that brought about by an automobile accident, fall, or knife or bullet wound, (ii) chemical injury, (iii) vascular injury or blockage, (iii) infectious or inflammatory injury such as that caused by a condition known as transverse myelitis, (iii) a tumor-induced injury, whether primary or metastatic or (iv) surgical injury. Thus, injuries leading to a state associated with diminished potential for axonal growth can be direct, e.g., due to concussion, laceration, or intrarnedullary hemorrhage, or indirect, e.g., due to extramedullary pressure of loss of blood supply and infarction.

Also provided herein are methods and compositions that will be useful in treating neurons in both the descending (e.g., corticospinal tract) and ascending tracts (e.g., the dorsal column-medial lemniscal system, the lateral spinothalarnic tract, and the spinocerebellar tract) of the spinal cord and in the reestablishment of appropriate spinal connections.

Common mechanisms of spinal cord injury include fractures of the vertebrae, which can damage the spinal cord from the concussive effect of injury due to displaced bony fragments, or damaged blood vessels, or contusion of emerging nerve roots. Dislocation of vertebrae can also cause spinal cord damage; dislocation is often the result of the rupture of an intervertebral disk, and may result in partial or complete severance of the spinal cord. Penetrating wounds can also cause severance or partial severance of the cord. Epidural hemorrhage and spinal subdural hematoma can result in progressive paraparesis due to pressure on the spinal cord. Examples of indirect injury to the spinal cord include damage induced by a blow on the head or a fall on the feet.

Intramedullary injury can be the result of direct pressure on the cord or the passage of a pressure wave through the cord, laceration of the cord by bone, or the rupture of a blood vessel during the passage of a pressure wave through the cord with a hemorrhage into the cord. Intramedullary bleeding and hematoma formation can also be caused by rupture of a weakened blood vessel. Ischemic damage can occur following compression of the anterior spinal artery, pressure on the anastornotic arteries, or damage to major vessels (Gilroy, in Basic Neurology, McGraw-Hill, Inc. New York, N.Y. (1990).

The methods and compositions described herein will also be useful in promoting the recovery of subjects with herniated disks, hyperextension-flexion injuries to the cervical spine and cervical cord, and cervical spondylosis.

In addition to treating movement disorders, the methods and compositions described herein may be used in treating disorders of the brain, e.g. the brain stem, and in enhancing brain or brain stem function in a subject with a state characterized by diminished potential for axonal growth. For example, the methods and compositions presented herein can be used in the treatment of brain damage. For example, the brain damage can be caused by stroke, bleeding trauma, or can be tumor-related brain damage.

The methods and compositions presented herein will also be useful in treating peripheral neuropathies. Damage to peripheral nerves can be temporary or permanent and, accordingly, the methods and compositions presented herein can hasten recovery or ameliorate symptoms. Peripheral neuropathies include, among others, those caused by trauma, diabetes mellitus, infarction of peripheral nerves, herniated disks, epidural masses, and postinfectious (or postvaccinal) polyneurites. The symptoms of peripheral neuropathies which will benefit from the methods and compositions presented herein include muscle wasting and weakness, atrophy, the appearance of fasciculations, impaired tendon reflexes, impaired sensation, dysethesias or paresthesias, loss of sweating, alteration in bladder function, constipation, causalgia, and male impotence.

Also provided are methods and compositions to treat neurodegenerative diseases, such as, Pick's disease, progressive aphasia without dementia, supranuclear palsy, Shy-Drager Syndrome, Friedreich's ataxis, olivopontocerebellar degeneration, vitamin E deficiency and spinocerebellar degeneration, Roussy-Levy Syndrome, Alzheimer's disease, Parkinson's disease, cancer, or viral infections, and hereditary Spastic ataxia or paraparesis. In addition, treatment of other disorders of the spinal cord, such as amyotrophic lateral sclerosis, spinal muscular atrophies, and multiple sclerosis are intended to be part of the methods and compositions presented herein. In other embodiments the methods and compositions presented herein will be useful in ameliorating the symptoms of neural degeneration such as that induced by vitamin B 12 deficiency, or associated with HIV infection (AIDS), or HTLV-I infection.

Other diseases that may treated include eye diseases or conditions which may benefit from axon regeneration or neural cell transplantation. Exemplary diseases include glaucoma, optic nerve severances, optic nerve neuritis, degeneration of retinal ganglion cells (RGCs) and their nerve fibers, degeneration of photoreceptor cells, retinitis pigmentosa, macular degeneration, and diabetic retinopathy.

Other states characterized by diminished potential for axonal growth or regeneration, or neural transplant integration which will benefit by the methods and compositions presented herein will be apparent to one of ordinary skill in the art.

The methods and compositions presented herein can be used to treat any mammal, such as primates, canines, ovines, bovines, felines, and horses. In embodiments of the methods and compositions presented herein is used to treat human subjects. Subjects may be fetuses, embryos, neonates or adults. For example, human subjects may be less than about 1, 2 or 3 months old; less than about 1, 2 or 3 years old.

Additional agents which create an “environment” favorable to axonal growth or cell replacement may also be added. Exemplary agents include trophic factors, receptors, extracellular matrix proteins, intrinsic factors, or adhesion molecules. Exemplary trophic factors include NGF, BDNF, NT-3, 4, 5, or 6, CNTF, LIF, IGFI, IGFII, GDNF, GPA, bFGF, TGFB, and apolipoprotein E. Exemplary receptors include the Trk family of receptors. An exemplary extracellular matrix protein is laminin. Exemplary intrinsic factors include GAP-43 (also known as B 50, pp46, neuromodulin, and F I), cAMP, and ameloid precursor protein (APP) (Moya et al. Del,. Biol. 161:597 (1994)). Exemplary adhesion molecules include NCAM and L 1. Nucleic acids encoding these polypeptides, or the polypeptides may be used. The use of peptide fragments of any of the above axonal growth enhancers could also be used.

Agents which provide an environment favorable to axonal growth or cell transplantation, i.e., a permissive environment, can be administered to a subject by a variety of means. In some embodiments, they may be injected, either locally or systemically. In other embodiments they can be incorporated into a gene construct. In certain embodiments such agents can be supplied in conjunction with nerve guidance channels as described in U.S. Pat. Nos. 5,092,871 and 4,955, 892. For instance, a pharmaceutical preparation of the compound can be introduced systemically, e.g. by intravenous injection. In other embodiments, the delivery of the compound can be more limited with introduction into the animal being quite localized, for example delivery can be targeted to a specific area, e.g., the site of nerve or spinal cord injury or cell transplant. In the brain, the injection can be intraventricular. To facilitate local delivery the compound can be introduced by stereotactic injection (e.g. Chen et al. PNAS 91: 3054-3057(1994)). The pharmaceutical preparation of the compound can contain an acceptable diluent, or can contain a slow release matrix in which the gene delivery vehicle is imbedded.

In one embodiment, an agent is administered at the site of a neural injury, e.g., a spinal cord injury. The agent may be administered with a syringe or a stent (e.g., coated stent) to the site of injury. Agents can also be administered at the site of the injury during reparative surgery. They can also be administered at the site where the bodies of the neural cells are from which the axons were severed. For example, two nerve endings can be brought within a certain distance from one another, e.g., within less than about 10 mm, preferably less than about 6 mm, 3 mm, 1 mm, 750 μm, 500 μm, 300 μm, 100 μm, 70 μm, 50 μm, 30 μm, 10 μm or less. Prior to, durin after bringing the nerve endings together, an amount of agent providing a permissive environment and/or optionally an agent stimulating axon regeneration is added to the site where the nerve endings are brought together. The agent(s) can be present in a matrix for permitting slow release of the lithium.

In another embodiment, one or more of the agents described herein are administered orally. For example, an agent that stimulates axon regeneration, such as lithium or salt thereof, may be administered orally. Exemplary doses include those that are administered for treating bipolar disease or other conditions that are commonly treated with lithium.

Exemplary salts of lithium that can be used to promote axon regeneration include lithium chloride, lithium acetate, lithium carbonate, lithium citrate and lithium sulfate. For example, lithium chloride (LiCl) can be administered to a subject having a state characterized by diminished potential for axonal growth. Numerous salts of lithium are commercially available, e.g., for treating certain manic-depressive illnesses. Compounds having structural similarities to lithium or a salt thereof can also be used. Such alternative compounds can be tested according to methods described herein. They may also be tested for their ability to increase bcl-2 expression. Lithium or analogs or salts thereof can be administered systemically or locally.

A severed axonal process can be directed toward the nerve ending from which it was severed by a prosthesis nerve guide which may contain an agent such as described herein, as, e.g. a semi-solid formulation, or which is derivatized along the inner walls of the nerve guidance channel. These agents may be administered simultaneously with a therapeutic composition described herein.

In certain embodiments of the methods and compositions presented herein, for example in the treatment of long-standing injury (e.g., when there has been significant colateral sprouting of a neural cell) it may be desirable to a combine a treatment described herein with a “pruning procedure” to remove rostral sprouting (Schneider, G. E. Brain. Bahav Evol. 8:73 (1973)).

Pharmaceutical compositions for use in accordance with the methods and compositions presented herein, e.g., compositions comprising astrotoxin or inhibitors or GAFP or Vim, may be formulated in a conventional manner using one or more physiologically acceptable carriers or excipients. Thus, the compounds and their physiologically acceptable salts and solvates may be formulated for administration, for example, by injection. For example, the compositions provided herein can be formulated for a variety of loads of administration, including systemic. Techniques and formulations generally may be found in Remminglons Pharmaceutical Sciences, Meade Publishing Co., Easton, Pa. For systemic administration, injection is preferred, including intramuscular, intravenous, intraperitoneal, and subcutaneous. For injection, the compositions provided herein can be formulated in liquid solutions, preferably in physiologically compatible buffers such as Hank's solution or Ringer's solution. In addition, the compounds or agents may be formulated in solid form and redissolved or suspended immediately prior to use. Lyophilized forms are also included.

The compositions may be formulated for parenteral administration by injection, e.g., by bolus injection or continuous infusion. Formulations for injection may be presented in unit dosage form, e.g., in ampules or in multi-dose containers, with an added preservative. The compositions may take such compounds as suspensions, solutions or emulsions in oily or aqueous vehicles, and may contain formulation agents such as suspending, stabilizing and/or dispersing agents. Alternatively, the active ingredient may be in powder form for constitution with a suitable vehicle, e.g., sterile pyrogen-free water, or saline before use. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Toxicity and therapeutic efficacy of such compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50.

Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosages for use in humans. For example, the dosage of such compositions lies preferably within a range that includes the ED50 with little or no toxicity. The dosage may vary within this range depending upon the dosage form employed and the route of administration utilized. For any compound used in the methods and compositions presented herein, the therapeutically effective dose can be estimated initially from cell culture assays. A dose may be formulated in animal models to achieve a circulating plasma or local tissue concentration range that includes the IC50 (i.e., the concentration of the test compound which achieves a half-maximal therapeutic effect, e.g., inhibition of symptoms) as determined in cell culture. Such information can be used to more accurately determine useful doses in humans. Levels in plasma or local tissue may be measured, for example, by high performance liquid chromatography.

The regimen of administration can also affect what constitutes an effective amount. The compositions presented herein can be administered in several divided dosages, as well as staggered dosages, can be administered daily or sequentially, or the dose can be continuously infused, or can be a bolus injection. For example, the compositions presented herein may be administered in doses ranging from 0.1 mg/kg, 1 mg/kg, 10 mg/kg, 100 mg/kg, or 1000 mg/kg.

Further, the dosages of the agent(s) can be proportionally increased or decreased as indicated by the exigencies of the therapeutic or prophylactic situation.

Another embodiment is a packaged drug for the treatment of a state associated with diminished potential for axonal growth or regeneration, or neural transplant integration, which includes astrotoxin or an analog thereof or an inhibitor of GFAP or Vim, packaged with instructions for treating a subject.

The “packaged drug” can include any of the compositions described herein. The term “instructions” as used herein is meant to include the indication that the packaged drug is useful for treating a state associated with diminished potential for axonal growthor regeneration, or neural transplant integration and optionally may include the steps which one of ordinary skill in the art would perform to treat a subject with such a state.

Another aspect of the methods and compositions presented herein relates to the use of intermediate filament, e.g., GFAP and Vim nucleic acids in “antisense” therapy. As used herein, “antisense” therapy refers to administration or in situ generation of oligonucleotide molecules or their derivatives which specifically hybridize (e.g., bind) under cellular conditions, with the cellular mRNA and/or genomic DNA encoding one or more of the subject GFAP and Vim proteins so as to inhibit expression of that protein, e.g., by inhibiting transcription and/or translation. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense construct of the methods and compositions presented herein can be delivered, for example, as an expression plasmid which, when transcribed in the cell, produces RNA which is complementary to at least a unique portion of the cellular mRNA which encodes GFAP and Vim proteins. Alternatively, the antisense construct is an oligonucleotide probe which is generated ex vivo and which, when introduced into the cell causes inhibition of expression by hybridizing with the mRNA and/or genomic sequences of GFAP and Vim genes. Such oligonucleotide probes are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668. With respect to antisense DNA, oligodeoxyribonucleotides derived from the translation initiation site, e.g., between the −10 and +10 regions of the GFAP and Vim nucleotide sequences of interest, are preferred.

Antisense approaches may involve the design of oligonucleotides (either DNA or RNA) that are complementary to GFAP and Vim mRNAs. The antisense oligonucleotides may bind to GFAP and Vim mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. (Wagner, R. (1994) Nature 372:333). Therefore, oligonucleotides complementary to either the 5′ or 3′ untranslated, non-coding regions of GFAP and Vim genes could be used in an antisense approach to inhibit translation of endogenous GFAP and Vim mRNAs. Oligonucleotides complementary to the 5′ untranslated region of the mRNA should include the complement of the AUG start codon. Antisense oligonucleotides complementary to mRNA coding regions are less efficient inhibitors of translation but could also be used in accordance with the methods and compositions presented herein. Whether designed to hybridize to the 5′, 3′ or coding region of GFAP and Vim mRNAs, antisense nucleic acids should be at least six nucleotides in length, and are preferably less that about 100 and more preferably less than about 50, 25, 17 or 10 nucleotides in length.

Regardless of the choice of target sequence, it is preferred that in vitro studies are first performed to quantitate the ability of the antisense oligonucleotide to quantitate the ability of the antisense oligonucleotide to inhibit gene expression. In one embodiment these studies utilize controls that distinguish between antisense gene inhibition and nonspecific biological effects of oligonucleotides. In another embodiment these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. 84:648-652; PCT Publication No. W088/09810, published Dec. 15, 1988) or the blood-brain barrier (see, e.g., PCT Publication No. W089/10134, published Apr. 25, 1988), hybridization-triggered cleavage agents. (See, e.g., Krol et al. (1988) BioTechniques 6:958-976) or intercalating agents. (See, e.g., Zon (1988), Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an α-anomeric oligonucleotide. An α-anomeric oligonucleotide forms specific double-stranded hybrids with complementary RNA in which, contrary to the usual b-units, the strands run parallel to each other (Gautier et al. (1987) Nucl. Acids Res. 15:6625-6641). The oligonucleotide is a 2′-0-methylribonucleotide (Inoue et al. (1987) Nucl. Acids Res. 15:6131-6148), or a chimeric RNA-DNA analogue (Inoue et al. (1987) FEBS Lett. 215:327-330).

Oligonucleotides of the methods and compositions presented herein may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988) Nucl. Acids Res. 16:3209, methylphosphonate olgonucleotides can be prepared by use of controlled pore glass polymer supports. (Sarin et al. (1988) Proc. Natl. Acad. Sci. U.S.A. 85:7448-7451), etc.

The antisense molecules can be delivered to cells which express GFAP or Vim in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

A recombinant DNA construct in which the antisense oligonucleotide may be placed under the control of a strong pol III or pol II promoter may also be used. The use of such a construct to transfect target cells in the patient will result in the transcription of sufficient amounts of single stranded RNAs that will form complementary base pairs with the endogenous GFAP and Vim transcripts and thereby prevent translation of GFAP and Vim mRNAs. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region, (Bemoist et al. (1981) Nature 290:304-310), the promoter contained in the 3′ long terminal repeat of Rous sarcoma virus (Yamamoto et al. (1980) Cell 22:787-797), the herpes thymidine kinase promoter (Wagner et al. (1981) Proc. Natl. Acad. Sci. U.S.A. 78:1441-1445), the regulatory sequences of the metallothionein gene (Brinster et al. (1982) Nature 296:39-42), etc. Any type of plasmid, cosmid, YAC or viral vector can be used to prepare the recombinant DNA construct which can be introduced directly into the tissue site. Alternatively, viral vectors can be used which selectively infect the desired tissue, in which case administration may be accomplished by another route (e.g., systematically).

Another method for decreasing or blocking gene expression of GFAP and Vim is by introducing double stranded small interfering RNAs (siRNAs), which mediate sequence specific mRNA degradation. RNA interference (RNAi) is the process of sequence-specific, post-transcriptional gene silencing in animals and plants, initiated by double-stranded RNA (dsRNA) that is homologous in sequence to the silenced gene. In vivo, long dsRNA is cleaved by ribonuclease III to generate 21- and 22-nucleotide siRNAs. It has been shown that 21-nucleotide siRNA duplexes specifically suppress expression of endogenous and heterologous genes in different mammalian cell lines, including human embryonic kidney (293) and HeLa cells (Elbashir et al. Nature 2001; 411(6836):494-8). Accordingly, translation of a gene in a cell can be inhibited by contacting the cell with short doublestranded RNAs having a length of about 15 to 30 nucleotides, preferably of about 18 to 21 nucleotides and most preferably 19 to 21 nucleotides. Alternatively, a vector encoding for such siRNAs or hairpin RNAs that are metabolized into siRNAs can be introduced into a target cell (see, e.g., McManus et al. (2002) RNA 8:842; Xia et al. (2002) Nature Biotechnology 20:1006; and Brummelkamp et al. (2002) Science 296:550). Vectors that can be used are commercially available, e.g., from OligoEngine under the name pSuper RNAi System™.

Ribozyme molecules designed to catalytically cleave GFAP and Vim mRNA transcripts can also be used to prevent translation of GFAP and Vim mRNAs and expression of GFAP and Vim polypeptides, or both (See, e.g., PCT International Publication WO90/11364, published Oct. 4, 1990; Sarver et al. (1990) Science 247:1222-1225 and U.S. Pat. No. 5,093,246). While ribozymes that cleave mRNA at site specific recognition sequences can be used to destroy GFAP and Vim mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach (1988) Nature 334:585-591. There are a number of potential hammerhead ribozyme cleavage sites within the nucleotide sequence of human GFAP and Vim cDNAs. Preferably the ribozyme is engineered so that the cleavage recognition site is located near the 5′ end of GFAP and Vim mRNAs; i.e., to increase efficiency and minimize the intracellular accumulation of non-functional mRNA transcripts.

The ribozymes of the the methods and compositions presented herein also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al. (1984) Science 224:574-578; Zaug, et al. (1986) Science 231:470-475; Zaug, et al. (1986) Nature 324:429-433; published International patent application No. WO88/04300 by University Patents Inc.; Been, et al. (1986) Cell 47:207-216). The Cech-type ribozymes have an eight base pair active site which hybridizes to a target RNA sequence whereafter cleavage of the target RNA takes place. The methods and compositions presented herein encompasses those Cech-type ribozymes which target eight base-pair active site sequences that are present in GFAP and Vim genes.

As in the antisense approach, the ribozymes can be composed of modified oligonucleotides (e.g., for improved stability, targeting, etc.) and should be delivered to cells which express GFAP and Vim genes in vivo. A preferred method of delivery involves using a DNA construct “encoding” the ribozyme under the control of a strong constitutive pol III or pol II promoter, so that transfected cells will produce sufficient quantities of the ribozyme to destroy endogenous GFAP and Vim messages and inhibit translation. Because ribozymes unlike antisense molecules, are catalytic, a lower intracellular concentration is required for efficiency.

Endogenous GFAP and Vim gene expression or expression of a splice form thereof can also be reduced by inactivating or “knocking out” GFAP and Vim genes or their promoter or a specific exon, using targeted homologous recombination. (E.g., see Smithies et al. (1985) Nature 317:230-234; Thomas, et al. (1987) Cell 51:503-512; Thompson et al. (1989) Cell 5:313-321; each of which is incorporated by reference herein in its entirety). For example, mutant, non-functional GFAP and Vim (or a completely unrelated DNA sequence) flanked by DNA homologous to the endogenous GFAP and Vim genes (either the coding regions or regulatory regions of GFAP and Vim genes) can be used, with or without a selectable marker and/or a negative selectable marker, to transfect cells that express GFAP and Vim in vivo. Insertion of the DNA construct, via targeted homologous recombination, results in inactivation of GFAP and Vim genes or splice forms thereof. Such approaches are particularly suited in the agricultural field where modifications to ES (embryonic stem) cells can be used to generate animal offspring with an inactive GFAP and Vim (e.g., see Thomas, et al. (1987) and Thompson (1989) supra). However this approach can be adapted for use in humans provided the recombinant DNA constructs are directly administered or targeted to the required site in vivo using appropriate viral vectors.

Nucleic acid molecules to be used in triple helix formation for the inhibition of transcription of GFAP and Vim genes are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

Other methods for rendering an environment permissive include contacting the environment with a dominant negative mutant of an intermediate filament protein, e.g., GFAP or vimentin. Yet other methods include contacting the environment with agents that reduce or inhibit the activity of an intermediate filament.

Antisense RNA and DNA, ribozyme, and triple helix molecules of the methods and compositions presented herein may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

In another embodiment, a nucleic acid encoding a polypeptide of interest, or an equivalent thereof, such as a functionally active fragment of the polypeptide or a dominant negative fragment of the polypeptide, is administered to a subject, such that the nucleic acid arrives at the site of the diseased cells, traverses the cell membrane and is expressed in the diseased cell.

Any means for the introduction of polynucleotides into mammals, human or non-human, may be adapted to the practice of the methods for the delivery of the various constructs into the intended recipient. In one embodiment, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. A colloidal system may be a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below).

In another method, the DNA constructs are delivered using viral vectors. The transgene may be incorporated into any of a variety of viral vectors useful in gene therapy, such as recombinant retroviruses, adenovirus, adeno-associated virus (AAV), and herpes simplex virus-1, or recombinant bacterial or eukaryotic plasmids. While various viral vectors may be used in the practice of the methods described herein, AAV- and adenovirus-based approaches are of particular interest. Such vectors are generally understood to be the recombinant gene delivery system of choice for the transfer of exogenous genes in vivo, particularly into humans.

It is possible to limit the infection spectrum of viruses by modifying the viral packaging proteins on the surface of the viral particle (see, for example PCT publications WO93/25234, WO94/06920, and WO94/11524). For instance, strategies for the modification of the infection spectrum of viral vectors include: coupling antibodies specific for cell surface antigens to envelope protein (Roux et al., (1989) PNAS USA 86:9079-9083; Julan et al., (1992) J. Gen Virol 73:3251-3255; and Goud et al., (1983) Virology 163:251-254); or coupling cell surface ligands to the viral envelope proteins (Neda et al., (1991.) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector.

The expression of or inhibition of the expression of a polypeptide of interest in cells of a patient to which a nucleic acid encoding the polypeptide or inhibiting expression was administered can be determined, e.g., by obtaining a sample of the cells of the patient and determining the level of the polypeptide in the sample, relative to a control sample.

Provided herein are methods of promoting cell replacement or neural transplantation, into a subject that would benefit from the replacement of cells. A variety of cells may be used as replacements cells, such as stem cells, e.g., totipotent or pluripotent, stem cells, e.g., from the placenta, liver, or bone marrow, or neuronally derived cells. Alternatively, the cells may be derived from primary cell cultures, especially primary cultures of neurons, or from propagated cell cultures. In one embodiment, placental derived stem cells that can be obtained from the amnion, chorion or decidual layers of the placenta are used.

In addition, placental derived stem cells have been found to be capable of differentiating into a variety of tissue types including but not limited to hematopoetic, liver, pancreatic, nervous and endothelial tissues. Such cells are particularly useful to restore function in diseased tissues via transplantation therapy or tissue engineering, and to study metabolism and toxicity of compounds in drug discovery efforts.

In one embodiment, placental derived stem cells or neuronally-derived cells may be transplanted directly into the recipient where the cells will proliferate and differentiate to form new tissue thereby providing the physiological processes normally provided by that tissue. Alternatively, placental derived stem cells may be transplanted as a differentiated cell population, such as neurons. Prior to injection or implantation into the target sites of the subject, the cells may be genetically modified to promote the differentiation or survival of certain neuronal or glial cell types, or to promote the formation of function synapses by the transplanted cells.

The replacement cells can be injected or implanted into target sites in the subjects, preferably via a delivery device, such as a tube, e.g., catheter, for injecting cells and fluids into the body of a recipient subject. In one embodiment, the tubes additionally have a needle, e.g., a syringe, through which the replacement cells can be introduced into the subject at a desired location. The replacement cells can be inserted into such a delivery device, e.g., a syringe, in different forms. For example, the replacement cells can be suspended in a solution or embedded in a support matrix when contained in such a delivery device. As used herein, the term “solution” includes a pharmaceutically acceptable carrier or diluent in which the cells remain viable. Pharmaceutically acceptable carriers and diluents include saline, aqueous buffer solutions, solvents and/or dispersion media. The use of such carriers and diluents is well known in the art. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions can be prepared by incorporating progenitor cells as described herein in a pharmaceutically acceptable carrier or diluent and, as required, other ingredients enumerated above, followed by filtered sterilization.

In another embodiment, the replacement cells may be attached in vitro to a natural or synthetic matrix that provides support for the cells prior to delivery to the subject. The matrix will have all the features commonly associated with being biocompatible, in that it is in a form that does not produce an adverse, or allergic reaction when administered to the recipient host. Agents that create a permissive environment, and optionally agents that stimulate axonal growth and optionally growth factors capable of stimulating the growth and regeneration of neurological tissue may also be incorporated into matrices. Such matrices may be formed from both natural or synthetic materials and may be designed to allow for sustained release of growth factors over prolonged periods of time. In yet another embodiment, it is contemplated that a biodegradable matrix that is capable of being reabsorbed into the body can be used.

To inhibit reactive astroglial cells near or in the vicinity of cell transplantation, astrotoxin or an analog thereof may be delivered to the area near or in the vicinity of the transplanted cells. For example, astrotoxin or an analog thereof may be delivered in doses ranging from about 0.1 mg/ml, 1 mg/ml, 10 mg/ml or 100 mg/ml solutions.

To improve replacement cell adhesion to the matrix and survival and function of the replacement cell, the matrix may optionally be coated in its external surface with factors known in the art to promote cell adhesion, growth or survival. Such factors include cell adhesion molecules, extra cellular matrix molecules or growth factors.

Also provided herein is the use of replacement cells in three dimensional cell and tissue culture systems to form structures analogous to tissue counterparts in vivo, such as various areas of the peripheral and central nervous system. The resulting tissue will survive for prolonged periods of time, and perform tissue-specific functions following transplantation into the recipient host. Methods for producing such structures is described in U.S. Pat. No. 5,624,840, which is incorporated herein in its entirety.

The present methods and compositions described herein may employ replacement cells derived from genetically-engineered cells to enable them to produce a therapeutic protein to treat a subject. As used herein the term “therapeutic protein” includes a wide range of functionally active biologically active proteins including, but not limited to, growth factors, cytokines, hormones, inhibitors of cytokines, peptide growth and differentiation factors.

One or more compositions described herein may be provided in the form of a kit. The kit may be a therapeutic kit. A kit may comprise, e.g., a composition comprising an agent that renders a cellular environment permissive to axon regeneration or cell transplant and/or a composition comprising an agent that promotes axon regeneration. Other components of the kit may include instructions for use, devices for administration of the agents, such as a syringe or a stent. A kit may comprise several doses of each of the agents.

The methods and compositions presented herein, now being generally described, will be more readily understood by reference to the following examples, which are included merely for purposes of illustration of certain aspects and embodiments of the methods and compositions presented herein and are not intended to limit the methods and compositions presented herein.

### EXAMPLES

### Example 1

### Bcl-2 Re-Establishes the Regenerative Potential of CNS Axons in Adult Mice

Potential growth obstacles within the CNS include myelin-associated inhibitory molecules and glial scarring after injury (5, 6). Successful regeneration in the adult CNS may require manipulating both the intrinsic features of injured neurons and the CNS environment.

To understand both the intrinsic features of injured neurons and the CNS environment that promote neural regeneration, an optic nerve regeneration model is useful. Rodent retinal ganglion cells (RGCs) lose their intrinsic ability to regenerate a severed optic nerve before birth (7, 8) and this loss precedes the onset of axonal growth inhibition in the CNS. Such a developmental scheme provides an opportunity to characterize independently the intrinsic and environmental mechanisms that regulate the regeneration of RGC axons.

One candidate is Bcl-2, an anti-apoptotic gene whose expression in primary neurons correlates with their ability to grow axons in culture (9-11). However, overexpression of Bcl-2 in P5 and adult transgenic mice fails to promote optic nerve regeneration in vivo (12, 13). Thus, it is not clear whether overexpression of Bcl-2 is insufficient to support the intrinsic growth mechanisms of RGC axons in vivo, or whether inhibitory mechanisms in the CNS environment block regeneration in P5 Bcl-2-transgenic (Bcl-2tg) mice.

Optic nerve crush was performed in Bcl-2tg (14) and wild-type littermate mouse pups, prior to the knowledge of genotypes. Regeneration was assessed and corroborated by three labeling methods: anterograde axon tracing, immunofluorescent staining, and retrograde labeling of RGCs that regenerated their axons. An anterograde tracer, cholera toxin B subunit conjugated with rhodamine (CTB-R) or fluorescein (CTB-F), was injected into the eye immediately after nerve was crushed. In sham-operated mice (n=6) and mice with incomplete optic nerve crush (n=4), CTB-R was transported along the undamaged axons to the lateral geniculate nuclei (LGN) within 24 h and reached its final target—the superior colliculus (SC)—within 48 h.

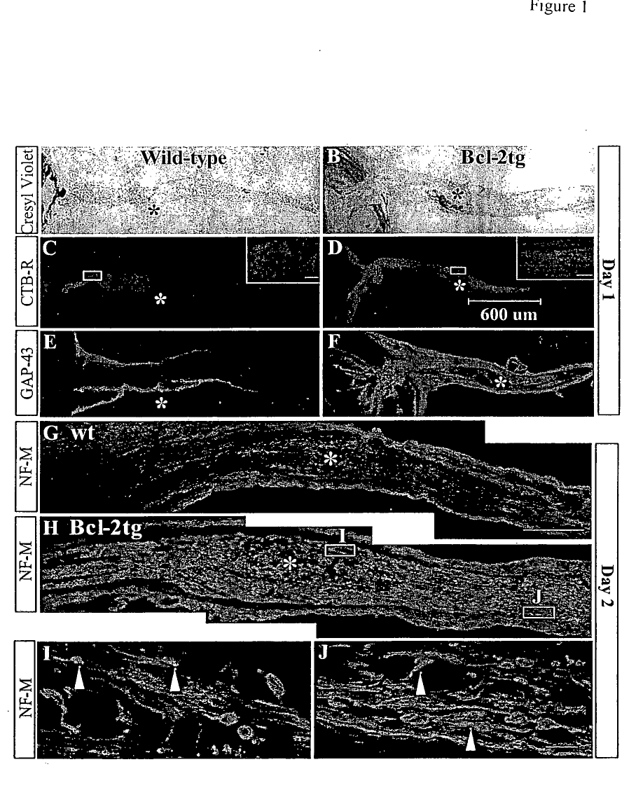

We first assessed optic nerve regeneration in P3 wild-type and Bcl-2tg mouse pups at 24 h after surgery (day 1). No regenerative response was observed in any of the wild-type mice (n=11) (FIG. 1A, C, E). CTB-R labeling stopped proximal to the crush site, and morphological analysis showed “bulb-like” structures, signifying ongoing axonal degeneration (FIG. 1C). Similar results were obtained by immunofluorescence staining for growth-associated protein 43 (GAP-43) (FIG. 1E) and medium-molecular -weight neurofilament protein (NF-M) (FIG. 1G), a marker of regenerating/live axons.

In all Bcl-2tg mice (n=8), however, large number of CTB-R labeled axons extended beyond the lesion site (FIG. 1d, f), indicating robust regeneration of severed axons. The crush site was identified by a traumatized zone containing degenerated cells and tissue debris (FIG. 1B) or by staining for isolectin, a marker of activated microglial cells (15)(data not shown). CTB-R-labeled axons appeared to be fasciculated and stopped 500-1000 mm caudal to the lesion (FIG. 1D). No labeling was seen beyond this point or in the brain sections, suggesting that these were not fibers spared from crush injury, which labeling would have passed through the optic chiasm into the brain at this time point. Similar patterns of axonal regeneration in Bcl-2tg mice were shown by immunofluorescence labeling of GAP-43 (FIG. 1F).

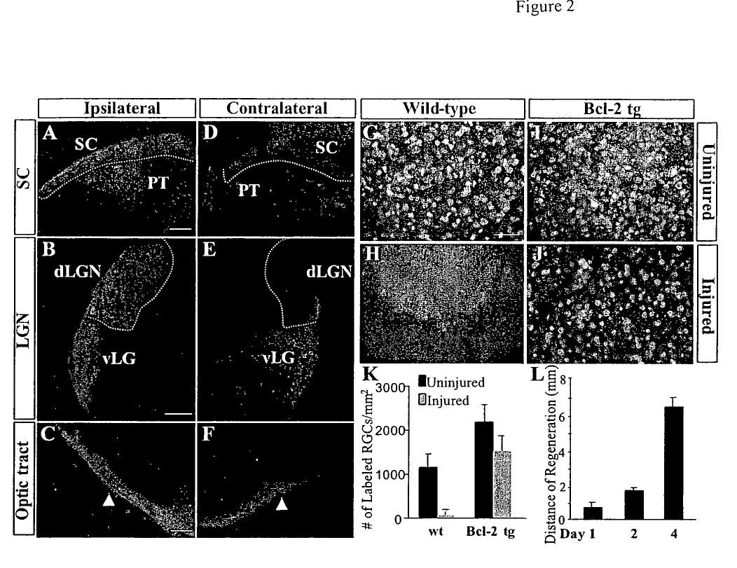

On day 2, all optic nerve sections from wild-type mice (n=19) contained only degenerating axons (FIG. 1g). However, in the Bcl-2tg mice, labeled axons extended further to 1000-2000 μm caudal to the lesion (n=5) (FIG. 1H-J), and many terminated in bullet-shaped structures resembling growth cones (FIG. 1I-K), again indicating active growth rather than mere survival after injury. By day 4 (n=12), regenerating axons had entered the brain in Bcl-2tg mice (FIG. 2A-F). Overall, from days 1-4, complete axonal degeneration were seen in 33 of 34 wild-type mice, whereas robust optic nerve regeneration—with thick bundles of axons passing beyond the lesion sites or reaching the brain—was seen in 24 (96.0%) of 25 Bcl-2tg mice.

Next, we examined CTB-F-labeled axon trajectories reconstructed in three dimensions in coronal brain sections. As expected, no CTB-F labeling was found in the brains of wild-type mice (n=7) or in Bcl-2tg mice on days 1 and 2 after injury. However, extensive CTB-F labeling was observed in the brain sections of Bcl-2tg mice on day 4 (n=6). Most labeled axons traveled along optic tract pathways and reached their midbrain targets, including the LGN, pretectal nuclei, and SC (FIG. 2A-F). Aberrant projections to areas outside of these pathways or targets were noticed occasionally. Surprisingly, in all cases examined, regenerating axons predominantly innervated their ipsilateral brain targets (FIG. 2A-F), which pattern of brain innervation mirrored that in sham-operated controls. These results further demonstrate robust regeneration, rather than mere survival of axons, after injury in Bcl-2tg mice and suggest that regenerating axons do follow guidance cues along the optic pathways and find the appropriate visual targets in the midbrain, although they reach the ispilateral side.

To determine the percentage of RGCs that regenerated their axons, we placed a retrograde tracer, FluoroGold, bilaterally into the SC (the distalmost brain target of the optic nerve) after nerve crush or sham operation and counted labeled RGCs (FIG. 2G-K). Because overexpression of Bcl-2 prevents the developmental loss of RGCs, these cells are more abundant in uninjured Bcl-2tg mice than in wild-type mice (15)(FIG. 2G, I, K). Eleven days after optic nerve crush, very few labeled cells were detected in the retinas of wild-type mice, consistent with their lack of axon regeneration (FIG. 2H, K). In contrast, numerous labeled RGCs, equivalent to approximately 70% of those from the uninjured eye, were counted in the retinas of Bcl-2tg mice (n=6) (FIG. 2J, K). The results indicate that the majority of RGC axons in Bcl-2tg mice regenerated over long distances and reached the SC.

In rodents, embryonic RGCs grow axons about ten times faster than postnatal RGCs, which exhibit a decreased intrinsic growth potential (0.5-2.0 mm/day versus 40-60 μm/day)(8, 16). To further assess the ability of Bcl-2 to maintain the intrinsic growth potential of RGC axons, we determined the speed of axon regeneration in Bcl-2tg mice (FIG. 2L). By subtracting the distance of axon regeneration measured on day 2 (1.0-2.0 mm) from that on day 1 (0.5-1.0 mm), we obtained a rate of 0.5-1.5 mm/day. Using an alternative method, we measured the full length of the optic nerve and added it to the length of the optic tract from the optic chiasm to the posterior border of the SC, in mice on day 4, when regenerating axons normally reached the SC. The total distance was 6-8 mm, yielding an average rate of 1.5-2.0 mm/day. Thus, Bcl-2 induced optic nerve regeneration at a rate of 0.5-2 mm/day, similar to that during embryonic stages (8, 16). Therefore, overexpression of Bcl-2 appears to prevent the loss of the intrinsic regenerative potential of neonatal RGC axons.

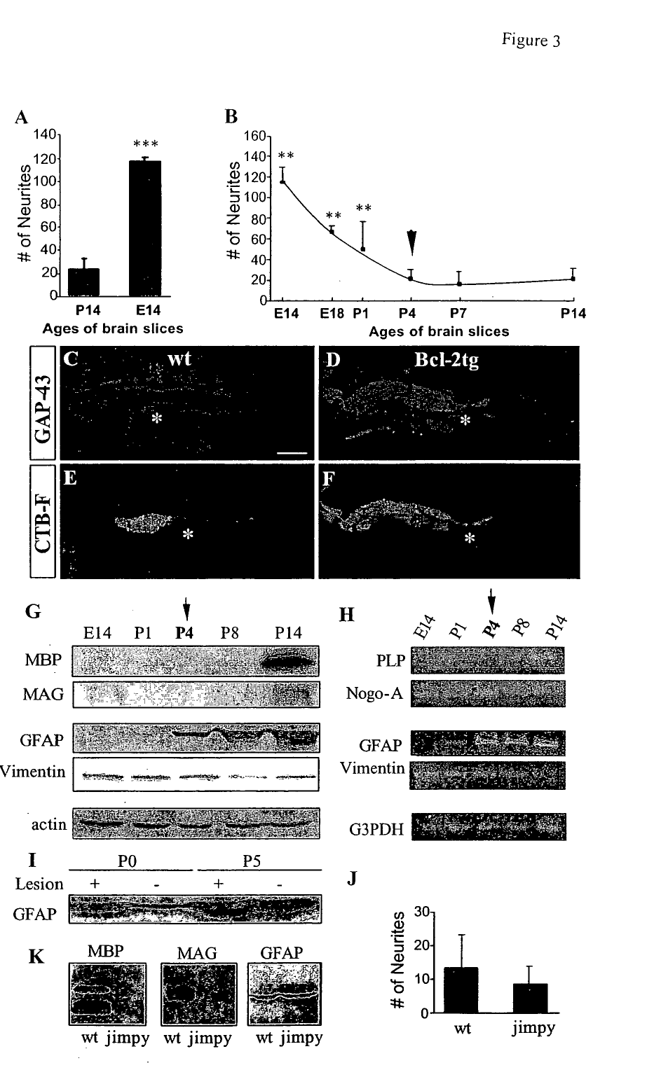

Next, we examined whether overexpression of Bcl-2 supports the intrinsic growth capacity of RGC axons up to adulthood. Using retina-midbrain slice co-cultures, we previously showed that retinas of adult Bcl-2tg mice grow axons into a permissive brain environment [e.g., a brain slice obtained on embryonic day 14 (E14)], suggesting that their RGCs maintain their intrinsic growth potential (9). We confirmed this in P14 retina-midbrain co-cultures, where the retina is populated with mature RGCs. A retinal explant from a P14 mouse was placed against a midbrain slice from an E14 or a P14 mouse (before and after the age of glial maturation). Consistent with their lack of intrinsic growth capacity, P14 wild-type retinal explants exhibited poor axonal growgrowth regardless of the age of the brain slice (data not shown). In contrast, P14 Bcl-2tg explants extended few axons into P14 brain slices, but grew robustly and innervated E14 brain slices, confirming that they can grow axons in a permissive environment (FIG. 3A).

To identify the mechanism that blocks optic nerve regeneration in Bcl-2tg mice and determine when exactly optic nerve regeneration fails in vivo, we performed optic nerve crush on P5 wild-type and Bcl-2tg mouse pups. In contrast to the robust regeneration in Bcl-2tg mice injured on P3, surviving fibers stopped anterior to the crush site and failed to grow in most Bcl-2tg mice injured on P5 (n=16). By day 4 in a few cases (n=2), labeled axons extended about 200 mm posterior to the crush site but not beyond (FIG. 3C-F). In all wild-type mice (n=15), few axons in optic nerve sections were identified by CTB-R or anti-GAP-43 labeling, even anterior to the crush site, indicating massive axon degeneration. None of the mouse brain sections, regardless of genotype, were positive for CTB-R. These findings are consistent with previous reports that overexpression of Bcl-2 fails to support optic nerve regeneration after P5 (12, 13).

To provide further evidence that the regenerative failure in P5 Bcl-2tg mice reflects a maturational change in the CNS environment, we set up a series of co-culture experiments. Retinal explants of P14 Bcl-2tg mice were placed against E14-P14 midbrain slices containing the SC. Growth inhibition in the midbrain environment increased with age and was maximal at P4; then it remained unchanged in older brains. Few axons from the P14 retinas of Bcl-2tg mice grew into brain slices of mice aged P4 or older, although as we showed previously, these RGCs retained the intrinsic growth capacity (FIG. 3B). These findings suggest that the growth inhibitory mechanisms in midbrain targets reach its adult level at around P4, which coincides with the induction of optic nerve regenerative failure in Bcl-2tg mice.

Next, we looked for correlated the onset of growth inhibition and developmental events in the midbrain SC, focusing on the formation of CNS myelin and the maturation of astrocytes (specifically, their ability to form glial scars), which have been proposed to contribute critically to this inhibitory mechanism (17, 18). Expression of three myelin markers—myelin basic protein (MBP), myelin-associated glycoprotein, and proteolipid protein—was not detectable in the SC at E14 to P8, long after the onset of midbrain growth inhibition (FIG. 3G, H). In contrast, expression of Nogo-A, an oligodendrocyte/myelin-associated protein, was detected in the SC as early as E14, when the midbrain environment is highly permissive for axon extension. These results are similar to those reported by others (19-21). The data suggest that the expression of myelin/oligodendrocyte-associated proteins does not correlate with the onset of CNS growth inhibition in vivo.

To further determine if CNS myelination affects the regrowth of retinal axons, we cultured retinal explants of P14 Bcl-2tg mice with brain slices from wild-type mice and jimpy mice, which are deficient in CNS myelin (FIG. 3k)(22). Interestingly, there was no significant difference in axonal invasion into wild-type and jimpy brain slices (n=3/group) (FIG. 3J), suggesting that CNS myelin does not prevent optic nerve regeneration in Bcl-2tg mice.

To assess the effects of astrocyte maturation in the midbrain, we examined the expression of vimentin (a marker of immature astrocytes) and glial fibrillary acidic protein (GFAP, a marker of mature astrocytes) (23, 24). GFAP expression increased in parallel with growth inhibition, being absent at E14, when the environment is permissive for growth, and rising steadily until reaching a plateau at about P4 (FIG. 3G, H). Conversely, vimentin expression in the SC was high at E14 and decreased thereafter (FIG. 3G, H). GFAP expression was also much higher in the midbrains of P5 mice than P0 mice before and after injury, indicating an enhanced ability of P5 astrocytes to become reactive after injury (FIG. 3I). Thus, growth inhibition appears to increase as astrocytes mature and acquire the ability to become reactive.

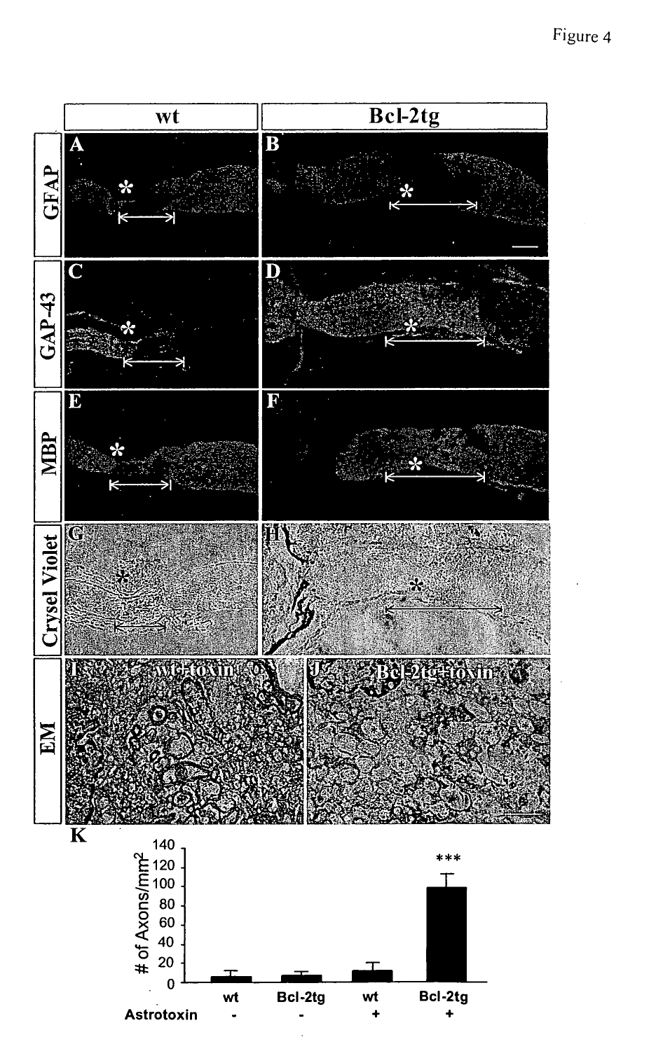

To assess the functional significance of astrocyte maturation in optic nerve regeneration, we treated adult (2-3-month-old) wild-type and Bcl-2tg mice with astrotoxin (L-alpha-aminoadipate), which selectively kills astrocytes and has minimal effects on surrounding neurons and myelin (25, 26). Immediately after optic nerve crush, gelform soaked in L-alpha-aminoadipate solution (10 mg/ml in PBS, Sigma) or PBS was placed against the crush site. As shown by immunofluorescence staining, astrotoxin created a GFAP-negative area in the optic nerve (FIG. 4A, B) but had no effect on myelin and neuronal processes (FIG. 4A-H). In the absence of astrotoxin, no sign of axonal regeneration was seen in wild-type or Bcl-2tg mice. CTB-R and immunofluorescence labeling showed that nerve fibers retracted in wild-type mice, and survived but failed to grow in Bcl-2tg mice (n=3). After astrotoxin treatment in wild-type mice, a few axons sprouted past the lesion site and grew into the GFAP-negative area (n=5)(FIG. 4A-C). In contrast, robust regeneration was seen in all Bcl-2tg mice treated with astrotoxin (n=5). Regenerating axons extended exclusively into the GFAP-negative area and stopped abruptly where GFAP staining reappeared (FIG. 4B, D). Unlike the expression of GFAP, in most optic nerve sections, intense MBP staining co-localized with regenerating axons (FIG. 4E, F), providing further evidence that CNS myelin does not necessarily obstruct axonal regrowth.

To quantify axonal regeneration, we used electron microscopy to examine optic nerve sections of adult wild-type and Bcl-2tg mice treated with astrotoxin or PBS. Degenerating myelin and tissue debris were found in sections of all groups (FIG. 4I, J). In treated Bcl-2tg mice (n=5), a large number of regenerating axons were seen 0.5 mm posterior to the crush site, an area devoid of astrocytes and their cellular processes (FIG. 4J). Most axons were unmyelinated, suggesting they were newly regenerated. In contrast, in other three groups, few axons were present in optic nerve sections if they were collected at 0.5 mm posterior to the crush (n=3/group). Regenerating axons were 20-fold more abundant in astrotoxin-treated Bcl-2tg mice than in PBS-treated wild-type mice (P<0.001) (FIG. 4K). Thus, elimination of mature astrocytes promoted robust optic nerve regeneration in adult Bcl-2tg mice.

To further assess the role of reactive astrocytes as key players in preventing optic nerve regeneration in vivo, we studied mice deficient in GFAP and vimentin (GFAP−/−Vim−/−), a model in which reactive astrocytes lack intermediate filaments and have a decreased ability to form compact glial scars (27). Otherwise, these mice have no obvious defects, and the development of their retinas, axonal projections, and optic nerve myelination appear to be normal (28). It has been shown that the retinal environment of GFAP−/−Vim−/− mouse is more permissive for transplanted neurons to grow neurites than that of wild-type mouse (28). Optic nerve crush was performed in wild-type, Bcl-2tg, GFAP−/−Vim−/−, and Bcl-2tgGFAP−/−Vim−/− mice on P5 or P14, after astrocytes have become more mature and growth inhibition in the midbrain has reached its peak level.

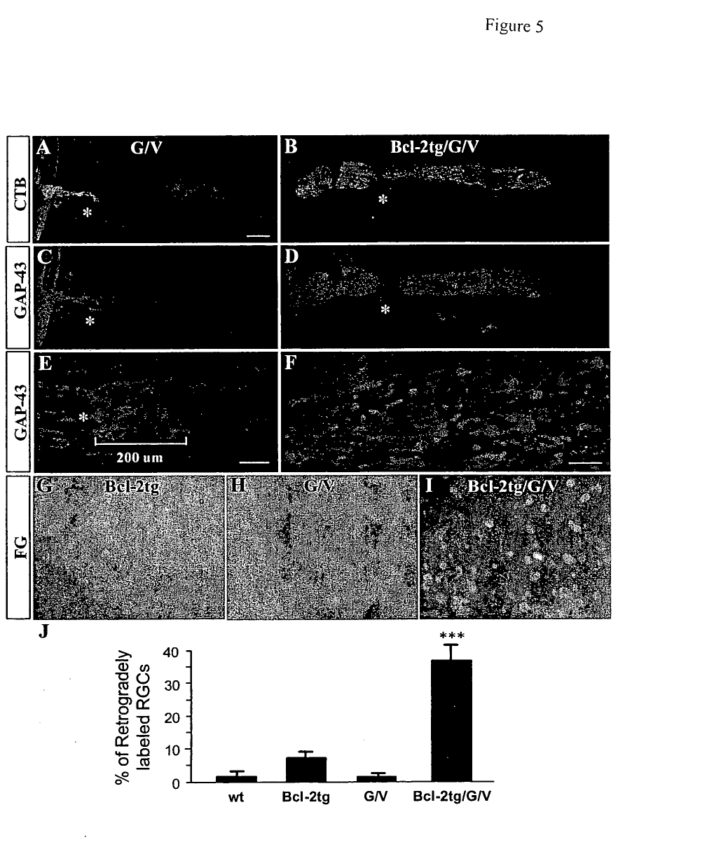

In wild-type mice, most axons degenerated and rapidly retracted after injury (P14, n=4). In Bcl-2tg mice, many severed axons survived, but they failed to regenerate and stopped anterior to the lesion site (P5, n=6; P14, n=3). Similar to that seen in astrotoxin-treated group, in GFAP−/−Vim−/− mice (P5, n=6), even those injured on P14 (n=4), many of the labeled axons grew past the lesion and extended 100-200 μm posterior to the crush site by day 4; the rate of axon extension was 25-50 μm/day, similar to that of the mature RGC axons (<60 μm/day). In contrast, severed axons regenerated rapidly and extensively in all Bcl-2tgGFAP−/−Vim−/− mice (P5, n=4; P14, n=4) (FIG. 5B, D) and had structures resembling growth cones at their tips (FIG. 5F). By day 4 after injury at both ages examined, labeled axons had extended the entire length of the optic nerve and innervated their brain targets—the LGN and SC—again, on the ipsilateral side. Aberrant projections to areas outside of the visual targets were more numerous in Bcl-2tgGFAP-−/−Vim−/− mice than in Bcl2tg mice injured on P3. Although the P14 optic nerves and brains of Bcl-2tgGFAP−/−Vim−/− mice were enriched of myelin in compared with those at P5, the intensity of the axon regeneration following injury appeared similar to those at P5.

To quantify the regeneration, we placed FluoroGold in the SCs of P5 wild-type , Bcl-2tg, GFAP−/−Vim−/−, and Bcl-2tgGFAP−/−Vim−/− mice immediately after optic nerve crush to label RGCs whose axons connected or regenerated to the SC. Since Bcl-2tg and Bcl-2tgGFAP−/−Vim−/− mice normally have more RGCs in the retina than wild-type and GFAP−/−Vim−/− mice, the number of RGCs with regenerated axons was expressed as a percentage of RGCs in the uninjured retina (FIG. 5J). Despite the increase in aberrant axonal projections after regeneration, approximately 30% of the severed axons reached the SC in Bcl-2tgGFAP−/−Vim−/− mice (n=3). In the three other groups, only a few cells were labeled (n=3/group). Thus, through their ability to become reactive and form scars after injury, mature astrocytes represent a key barrier to optic nerve regeneration in older Bcl-2tg mice.

The results of these studies demonstrate two parallel mechanisms that regulate optic nerve regeneration: an intrinsic developmental program for axon elongation supported by Bcl-2 and growth inhibition mediated by reactive astrocytes. Increasing Bcl-2 expression in the CNS while simultaneously suppressing reactive gliosis restored the regenerative potential of mature RGC axons and led to rapid, robust regeneration of severed optic nerves over long distances. Mouse RGCs lose Bcl-2 expression—and with it the intrinsic mechanisms that support axonal growth—around E18 (7, 9). Our findings show that overexpression of Bcl-2 is sufficient to maintain the regenerative potential of RGC axons up to adult and allows robust regeneration of severed CNS axons in vivo in a permissive brain environment. Expression of Bcl-2 restores the growth rate of regenerating axons of postnatal RGCs to values characteristic of embryonic life, and this suggests a novel function of Bcl-2 in addition to its regulation of apoptosis. Although the exact mechanisms are unclear, we demonstrate that Bcl-2 is an essential component for promoting CNS axon regeneration in vivo.

Our findings also show that reactive astrocytes, rather than myelin, inhibit axon regeneration in the adult CNS. These data support the previous reports that the failure of neurite outgrowth by transplanted neurons in vivo is directly associated with reactive glial cells, while adult myelinated white matter tracts appear to be highly permissive for regeneration (29, 30). Here, we show that maturation of astrocytes after P4 coincided with the failure of optic nerve regeneration. Genetic suppression of glial scar formation (27) or application of astrotoxin to destroy astrocytes without disrupting CNS myelin (25, 26) eliminated the growth barrier presented by the CNS environment and supported vigorous optic nerve regeneration in Bcl-2tg mice aged P4 or older. Even in a permissive environment (astrotoxin-treated or in GFAP−/−Vim−/−), mature RGCs had only a limited growth ability or grew slowly past the lesion site. In contrast, in Bcl-2tgGFAP−/−Vim−/− mice, RGC axons regenerated robustly and rapidly at a speed of embryonic axon elongation. The results confirm again a role of Bcl-2 in regulating the intrinsic growth potential of RGC axons. Further these results show that reactive astrocytes regulate the CNS environmental effect on CNS regeneration in mammals.

### Example 2

### Inactivation of Astrocytes in GFAP/Vim Deficient Mice Promotes Robust Neural Integration of Retinal Transplants

Retinal glial cells may constitute a barrier demarcating the mature host retina from transplanted cells (9′,10′). There are two types of glial cells in the retina: astrocytes and Müller cells. Astrocytes are located in the inner surface at the ganglion cell layer (GCL), while Müller cells have processes spanning from the inner retina to the outer limiting membrane and support retinal structures lying in between. After retinal transplantation, both types of glial cells become reactive and upregulate intermediate filament (IF) proteins—glial fibrillary acid protein (GFAP; GenBank Accession No.'s: NM_{—}002055, S40719 and NP_{—}002046) and vimentin (Vim; GenBank Accession No.'s: CD579735, CD579639, CD579486, CD579445, CD579260, AAH30573 and AAH00163) (11 ′,12′). It has been suggested that glial scarring after neural injury obstructs axonal regrowth by forming physical and diffusion barriers that separate the intact regions of the retina and CNS from the damaged area (9′,10′,13)′.

GFAP and vimentin form IFs, a part of the cytoskeleton, in astrocytes and Müller cells; IF production increases in reactive astrocytes under pathological conditions and after transplantation in the brain and the retina (14′,15′,16′). Increased production of IFs reportedly is associated with the formation of glial scar that is obstructive to axonal growth (16′,17′). Reactive astrocytes in GFAP−/−Vim−/−14′,18′, but not in GFAP−/−19′-21′ or Vim−/−22′, are completely devoid of IFs, and this leads to reduced glial scarring after CNS injury.

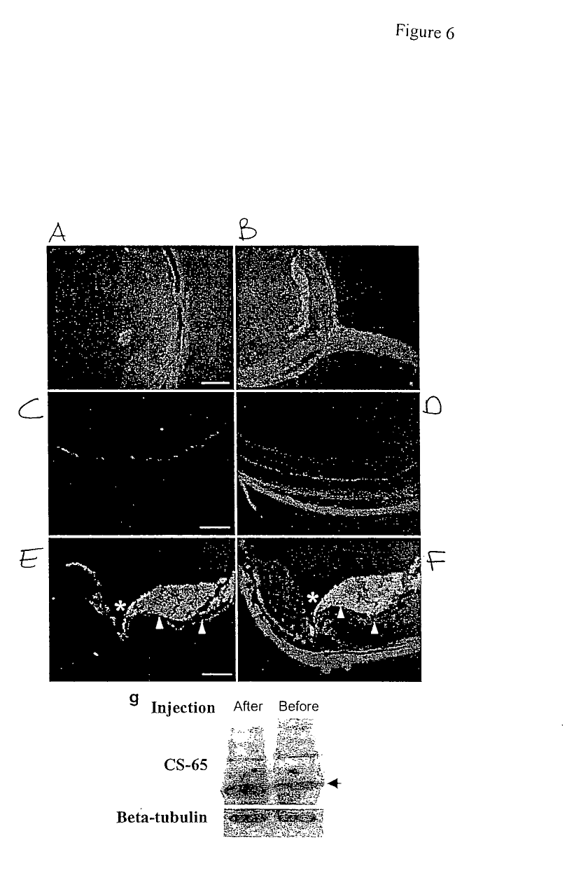

To distinguish implanted cells and their cellular processes from the host environment, we used retinas from mice expressing an enhanced green fluorescent protein (EGFP) transgene driven by a chicken b-actin promoter and cytomegalovirus enhancer (24′). Suspensions of retinal cells from postnatal day 0 (P0) EGFP mice were injected into the subretinal space of adult wild-type or GFAP−/−Vim−/− hosts. Three weeks after transplantation, most surviving donor-derived cells in the wild-type mice remained around the injection site, formed aggregates, and failed to migrate or extend neurite into the host retina (n=11) (FIG. 6A). To confirm that this failure of neural integration into the wild-type hosts was not a result of the site of transplantation, we injected cells into the vitreous cavity (n=4) (FIG. 6B). Again, the graft failed to integrate. Few neurons migrated and extended neurites into the damaged retinal area but not beyond (FIG. 1B). In both situations, within 24 h to 3 weeks after transplantation, the region around the transplantation site showed increased expression of GFAP and vimentin, indicating reactive gliosis (FIG. 6C-F). Production of chondroitin sulfate proteoglycan, another characteristic of reactive gliosis (25′, 26′), was also upregulated in the host retina (FIG. 6G).

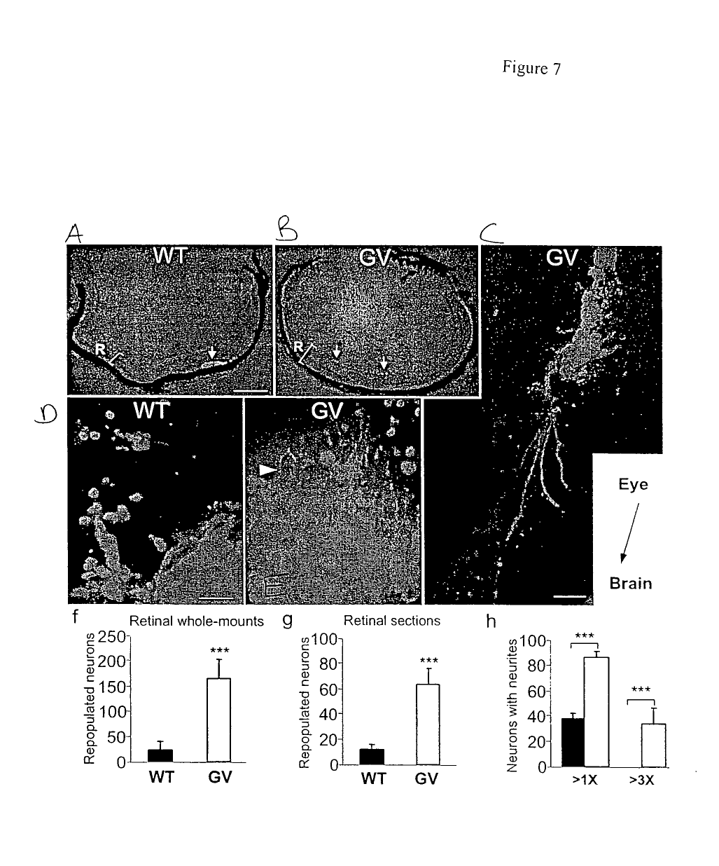

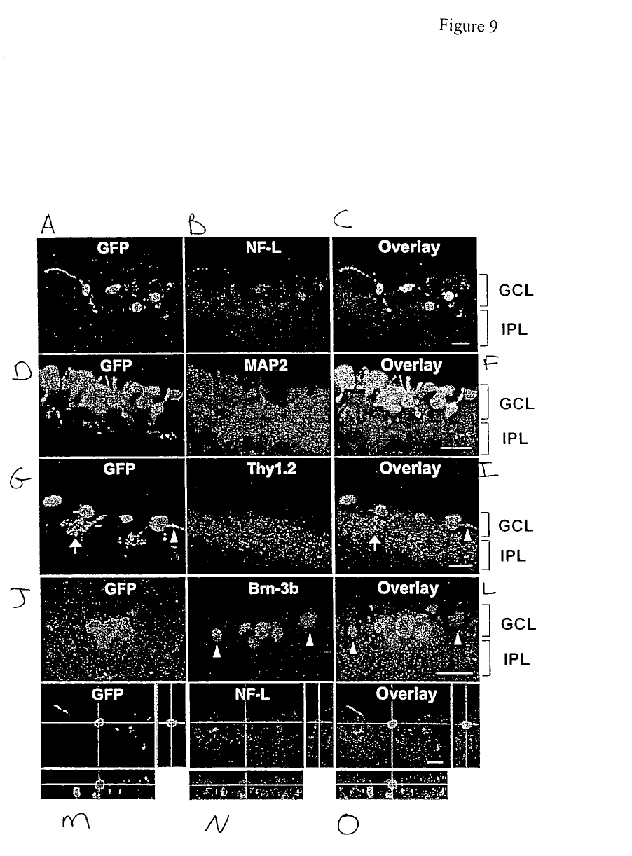

To determine whether reactive gliosis plays a role in preventing neural integration after transplantation, we injected retinal cells of P0 EGFP mice into the subretinal space of GFAP−/−Vim−/− mice (n=17). In contrast to the limited integration in the wild-type hosts (FIG. 7A), by 3 weeks after transplantation, numerous grafted cells had migrated away from the injection site and spread widely into the GCL in GFAP−/−Vim−/− hosts (FIG. 7B). Moreover, cells integrated into the GCL grew extensive neurites (FIG. 7E), primarily, into the inner plexiform layer (IPL) and the nerve fiber layer of the host retina (FIG. 9). In 3 of the 7 GFAP−/−Vim−/− mice examined, neurites were seen growing inside the optic nerve, reaching up to ˜1,000 μm posterior to the optic nerve head (FIG. 7C). At 6 months after transplantation, cells grafted into GFAP−/−Vim−/− retinas remained alive and well-integrated (n=3).

To quantify repopulation of the host retina, we counted cells that had spread out of the transplanted cell clusters at 3 weeks after transplantation. In both retinal whole-mounts and retinal sections, the number of migrating cells was consistently over six fold higher in GFAP−/−Vim−/− (whole-mounts: n=11; sections: n=3) than in wild-type hosts (whole-mounts: n=7; sections: n=4)(FIG. 7F, G). On average, grafted cells in wild-type hosts spread up to 484±129 μm from the injection site (n=3); whereas, those transplanted into GFAP−/−Vim−/− hosts spread throughout the retina, migrating up to 2,241±63 μm from the injection site (n=4)(P<0.005). We then assessed neurite outgrowth from the grafted cells in retinal whole-mounts. Most (˜90%) of the grafted cells in GFAP−/−Vim−/− retinas grew neurites, and about 30% of them had extended long neurites (>3 cell body lengths)(n=11)(FIG. 7H). In contrast, no grafted cells extended long neurites in the wild-type hosts (n=7) (FIG. 7H). These findings demonstrate that a specific change in glial properties of the retina of GFAP−/−Vim−/− mice permits neural graft integration and neurite outgrowth.

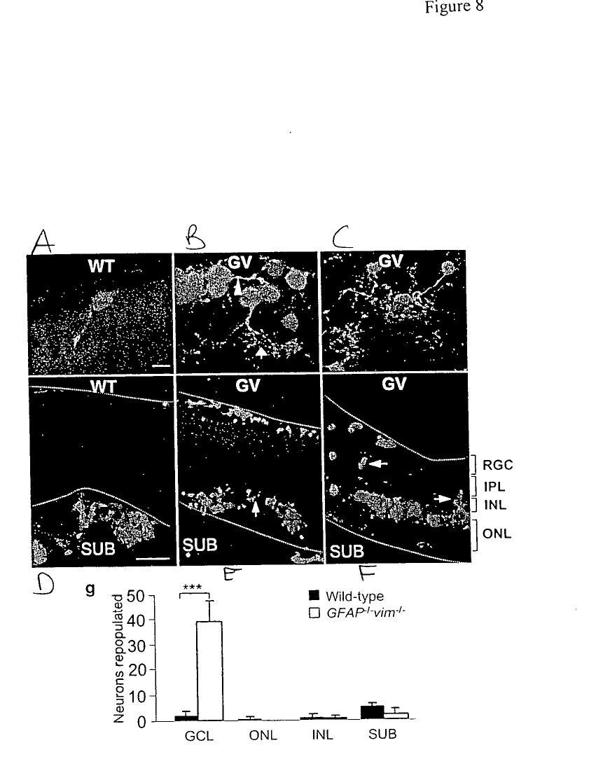

Developing P0 retinas are rich in immature neurons and neural precursor cells (27′). To determine whether the retinal environment of GFAP−/−Vim−/− mice would support the integration of cells from more mature retinas, we used retinas from P14-21 EGFP donor mice. At this stage, mouse retinas contain primarily post-mitotic and terminally differentiated neurons and have relatively few neural progenitor cells (27′,28′). After subretinal transplantation, robust neural integration was again observed into the retinas of GFAP−/−Vim−/− (n=7) but not wild-type (n=4) hosts (FIG. 8A-C). Therefore, retinal environment in GFAP−/−Vim−/− mice supports neural graft integration and neurite regeneration-even of transplants from retinas containing predominantly mature neurons.

Next, we asked whether transplanted cells integrated into different layers of the host retinas in GFAP−/−Vim−/− mice. Grafted cells that had integrated into the different layers of the retina were counted. The number of EGFP-positive cells that had migrated out of the transplanted cell clusters into the GCL was 25-fold greater in GFAP−/−Vim−/− hosts (n=3) than in wild-type hosts (n=4) (37.2±9.9 vs. 1.5±0.6 cells/10 retinal sections) (FIG. 8G). Success integration of grafted cells in GFAP−/−Vim−/− hosts appeared to occur primarily in the GCL and coincided with the presence of astrocytes in the retina (FIG. 8G).

To study the migration of grafted cells, we prepared retinal sections from mice 3, 7, 14, and 21 days after transplantation. Between days 3 and 14, numerous EGFP-positive cells were found between the outer retina and the GCL in GFAP−/−Vim−/− mice (n=7) (FIG. 8E, F), indicating neural migration from the subretinal space into the GCL. By day 21, most EGFP-positive cells had reached their final destination-the GCL-in GFAP−/−Vim−/− mice (n=5). In wild-type mice, however, few EGFP-positive cells were seen in-the retina; most remained around the injection site in the subretinal space and formed microaggregates (n=11) (FIG. 8F). Thus, a specific change in the microenvironment of the host retina enables grafted cells to migrate from the subretinal space and integrate into the GCL in GFAP−/−Vim−/− mice.

Remarkably, transplanted cells that integrated into the GCL of GFAP−/−Vim−/− hosts were morphologically similar to retinal ganglion cells (RGCs). We noted that many grafted cells elaborated complex dendrite-like tree structures in the inner plexiform layer (IPL) and a single axon-like process that entered the nerve fiber layer (FIG. 9). Since normal RGCs send only dendrites with numerous branches into the IPL but grow axons into the nerve fiber layer without elaboration, we referred the similar neurite morphology of grafted cells to dendrite- or axon-like processes, accordingly. Such neuronal architecture was not found in cells transplanted into wild-type hosts. There, only few cells migrated away from the injection sites into the GCL and occasionally grew short neurite-like processes that rarely branched (FIG. 8A). We estimated the dendrite-like growth of grafted cells in wild-type and GFAP−/−Vim−/− hosts by counting EGFP-positive cells that extended neurites into the IPL with at least one elaboration. The percentage of such cells was nine fold higher in GFAP−/−Vim−/− hosts (n=5) than in wild-type hosts (n=3)(50.7±14.3% vs. 5.6±6.9%, P<0.001), suggesting that the retinal environment in GFAP−/−Vim−/− mice supports the differentiation of dendritic morphology of transplanted cells.

Neuronal identity of integrated cells. To further characterize the grafted cells that survived and integrated into the host retinas, we stained retinal sections with antibodies against neuronal marker—low molecular weight neurofilament protein (NF-L) and microtubule-associated protein 2 (MAP2)—and glial marker, GFAP. At 21 days after transplantation, more than 85% of EGFP cells were found in the GCL of GFAP−/−Vim−/− host retinas, and most of their cells were positive for NF-L (FIGS. 7A-C and M-O) or MAP2 (FIG. 9D-F). Only a few EGFP cells were positive for GFAP or vimentin, suggesting they were astrocytes or Müller cells. We then stained the retina with antibodies against RGC-specific markers, Bm-3b29 and Thy1.230. Anti-B m-3b stains specifically RGC cell bodies, while anti-thy1.2 strongly labels dendritic (IPL) and axonal processes of RGCs. Examination by confocal microscopy confirmed that numerous EGFP-positive cells were positive for Bm-3B (FIG. 9J-L). Many of these cells extended a single axon-like process into the nerve fiber layer and branched dendritic processes into the IPL (FIG. 9G-I). Our results strongly indicate that, in the permissive environment of GFAP−/−Vim−/− hosts, the transplanted cells integrated into the host retina with distinct neuronal identity and established appropriate neuronal projections.

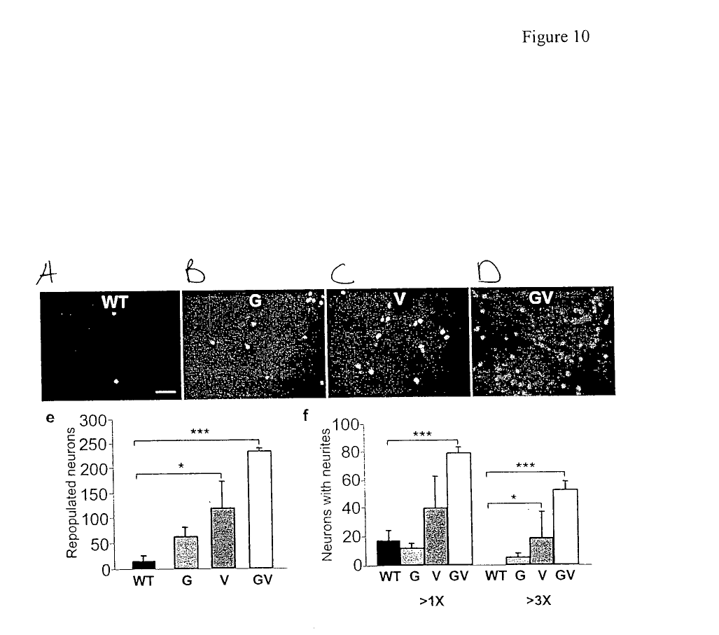

To distinguish whether the permissive environment for graft integration in GFAP−/−Vim−/− mice was caused by the absence of GFAP, vimentin, or both, we compared the outcome of retinal transplantation in wild-type, GFAP−/−, Vim−/−, and GFAP−/−Vim−/− mice in retinal whole-mounts (FIG. 10). Compared with wild-type hosts (n=4), GFAP−/− (n=4) and Vim−/− (n=4) mice had modest increases both in EGFP-positive cells that migrated outside the injection site and in labeled cells bearing neurites (>3-cell-body lengths). However, transplantation was vastly more successful in GFAP−/−Vim−/− hosts (n=6), as indicated by the much greater numbers of EGFP-labeled cells that migrated and extended neurites (FIG. 10). These results show that a partial deficiency in IFs in reactive astrocytes in mice (14′,31′) might improve the integration of retinal transplants. However, complete deficiency is required for the maximal effect.

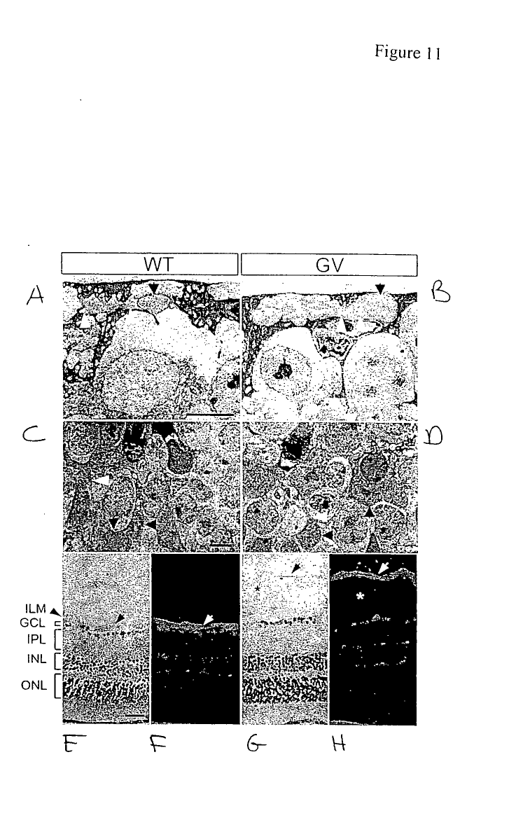

Morphology and function of the GFAP−/−Vim−/− retinas To preclude the possibility that mice deficient in GFAP and vimentin have other retinal abnormalities that contribute to graft integration, we examined retinal histology (n=3) and visual functions (n=3) of GFAP−/−Vim−/− mice. The GFAP−/−Vim−/− retinas had normal morphology, as shown by light and electron microscopy (n=3), and normal electrophysiological function (data not shown). The morphology of Müller cells also appeared normal, and their cellular processes spanned the entire retina. There were no signs of retinal degeneration. Examination by electron microscopy (n=3) confirmed that reactive astrocytes in the retinas of GFAP−/−Vim−/− mice, like those in other parts of the CNS14, were devoid of IFs (FIG. 6A, B). After subretinal injection, reactive Müller cells in the retinas of GFAP−/−Vim−/− mice had fewer cellular processes and also lacked IFs (FIG. 11C, D).

Interestingly, after removal of the lens in paraffin-embedded or frozen retinal sections from GFAP−/−Vim−/− mice (n=6), the inner limiting membrane (ILM) of the retina was consistently detached (FIG. 6E-H). This was not observed in intact eyeballs, even when examined by electron microscopy (n=3) (FIG. 11A, B), suggesting that the detachment resulted from tissue processing. To exclude the possibility that this change in the property of the ILM in GFAP−/−Vim−/− mice affected the fate of retinal transplants, we treated wild-type retinas with collagenase (100 U/ml) to break down the ILM (n=4). By comparing with untreated wild-type hosts (n=3), we found no difference in graft cell migration and neurite extension into the retinas of collagenase treated and untreated mice (data not shown). These results suggest that reactive astrocytes and Müller cells, but not the integrity of the ILM, present a barrier to the integration of transplanted cells into the retina.

It has been proposed that reactive and hypertrophic Müller cells constitute a barrier demarcating the mature host retina from subretinally transplanted cells (9′, 10′). For example, transplanted hippocampus-derived neural progenitor cells differentiate into neurons and glial cells and incorporate into the neonatal retina, but transplantation into the adult retina fail consistently (6′,32′). The elaboration and maturation of Müller cell processes and the migration of astrocytes into the retina have been suggested to contribute critically to this inhibition. Moreover, mechanically injured adult retinas or retinas exposed to transient ischemia support a limited extent of integration of transplanted stem cells (33′,34′). The most successful transplantations of hippocampal stem cells into mechanically injured retinas show that up to 25% of the surviving stem cells assume neuronal identities and develop synapse-like structures, at least transiently (33′). This happens within a region about 10-fold larger than the injury itself and coincides with increased GFAP immunoreactivity in both astrocytes and Müller cells (33′). In contrast, less than 1% of the transplanted stem cells integrates into the normal uninjured retinas (8′). These results suggest that retinal injury and the resulting regional change in the properties of astrocytes and Müller cells allow at least some degree of migration and integration of transplanted cells. Here, we showed that the integration of the transplants into the adult retina could be vastly improved without any trauma (except for the transplantation itself) when reactive astrocytes and Müller cells were depleted of IFs.

Glial scar formation may obstruct axonal regeneration in the mammalian CNS (13′,35′,36′). Since the absence of IFs in astrocytes allows better integration of retinal transplants, we might ask whether CNS regeneration would also be supported in GFAP−/−Vim−/− mice. Currently, no in vivo data are available on whether the CNS environment of GFAP−/−Vim−/− mice supports more robust axonal regeneration. Two groups have addressed the role of IFs of astroglial cells in neurite outgrowth in vitro (23′,37′,38′). One reports that GFAP−/−Vim−/− and GFAP−/− astrocytes are a better substrate for neurite outgrowth in vitro than wild-type astrocytes (23′,38′). The other finds comparable neurite outgrowth from wild-type and GFAP−/− astrocytes (37′). The latter finding is also compatible with the normal axonal sprouting and regeneration seen after dorsal hemisection of the spinal cord in GFAP−/− mice (39′). We observed that the absence of GFAP or vimentin alone either did not change or enhanced only moderately the migration and neurite extension of transplanted cells. These findings suggest that complete deficiency of IFs in astrocytes is required for the maximal effect on axonal regeneration in the CNS.

### Example 3

### Robust Optic Nerve Regeneration with Astrotoxin and Lithium in Adult Mice

Unlike the peripheral nervous system, axons in the adult CNS of mammals are unable to regenerate after injury. This regenerative failure has been attributed to both the hostile environment of CNS glial cells and the lack of intrinsic growth ability by CNS axons. Recently, growth inhibitory molecules have been identified from the CNS glial cells. These include chondroitin sulfate proteoglycans, ¹″ NogoA, myelin associated glycoprotein and oligodendrocyte-myelin glycoprotein.²″ However, depletions of Nogo or its receptor, NogoR and P75, fail to support CNS regeneration in vivo (Kim et al. (2003) Neuron 38:87). Recent evidence has suggested a critical role of reactive astrocytes in the failure of CNS regeneration.³− The failure of neurite outgrowth from transplanted neurons in vivo is associated directly with the presence of reactive glial cells, while adult myelinated white matter tracts can be highly permissive for the growth. Nevertheless, despite of the numerous attempts that have been made to neutralize the inhibitory effects of the mature CNS, only limited amount of axonal regrowth has been achieved in adult mammals.⁴″

Emerging evidence now indicates that mature CNS neurons also lack intrinsic mechanisms supporting axonal elongation. CNS neurons, such as RGCs, lose their intrinsic capacity to extend axons before birth. ⁵″ Previously, we have shown that overexpression of Bcl-2 in transgenic mice supports axonal regeneration in the neonatal stage in vivo.⁶″ Nevertheless, no signs of CNS regeneration are found in mature Bcl-2 transgenic mice.⁸″ A possible argument is that overexpression of Bcl-2 protein in mice delays neuronal maturation, and as a consequence, prolongs the window for CNS axon regeneration. However, our previous studies have suggested that RGCs overexpressing Bcl-2 retain the intrinsic capacity to grow axon throughout life (Chen et al. (1997) Nature 385:434). It thus raises the question whether the mature CNS environment develops growth inhibitory mechanisms in the postnatal stage and further blocks optic nerve regeneration in adult Bcl-2 transgenic mice. Therefore, for regeneration to occur in adult mice, it may be essential to use combined approaches that activate Bcl-2-supported growth mechanisms and suppress the functions of growth inhibitors in the CNS environment.

In the present study, we set out to test the effects of Bcl-2 and reactive astrocytes on optic nerve regeneration by taking a pharmacological approach. Although transgenic and knockout mice provide excellent genetic model systems to serve as a proof of principle, ideally, therapy for optic nerve injury should be accomplished with application of small molecular drugs. Previously, we have shown that a long standing mood stabilizer, lithium, induces Bcl-2 expression in the mouse retina and promotes retinal axon regeneration in vitro via a Bcl-2-dependent mechanism.⁷″ Lithium has also been reported to induce Bcl-2 expression in other CNS neurons (Chen et al. (1999) J Biol Chem. 274:6039; Chen et al. (1999) Neurochem. 72:925). In addition, astrotoxin—L-α-aminoadipate, a glutamate analogue—is known to selectively enter astrocytes and induce cytotoxicity, but it poses minimal effects on surrounding neurons and myelin. ^{9″-11″} Here, we asked whether the combined treatments of lithium, that induces Bcl-2 expression, and astrotoxin, which eliminates mature astrocytes and suppresses reactive gliosis, supported optic nerve regeneration in adult mice. This study may provide a therapeutic strategy for treating optic nerve and CNS damage in human.

Induction of Bcl-2 Expression by Lithium in the Mouse Retina

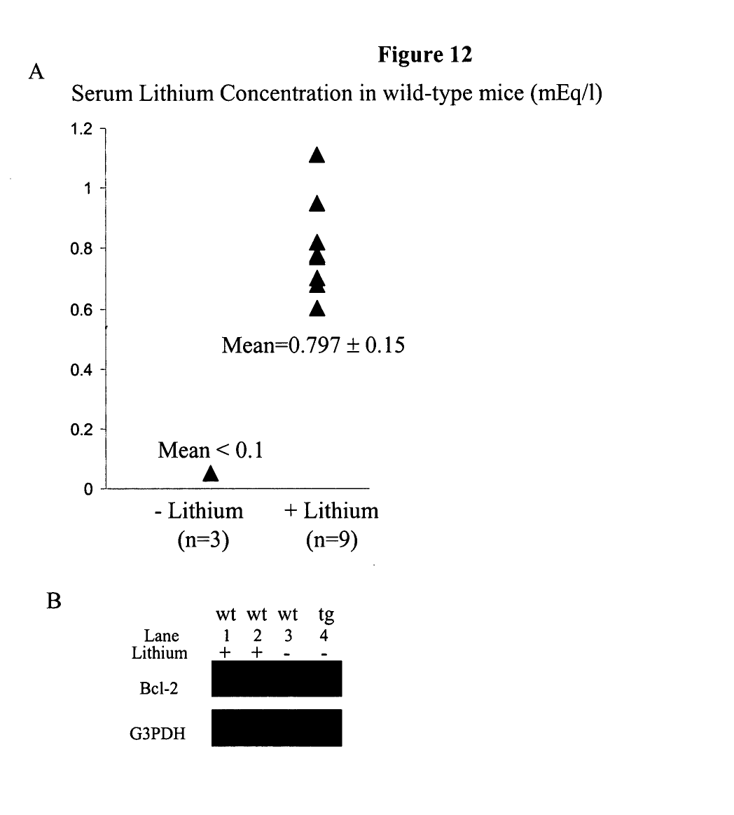

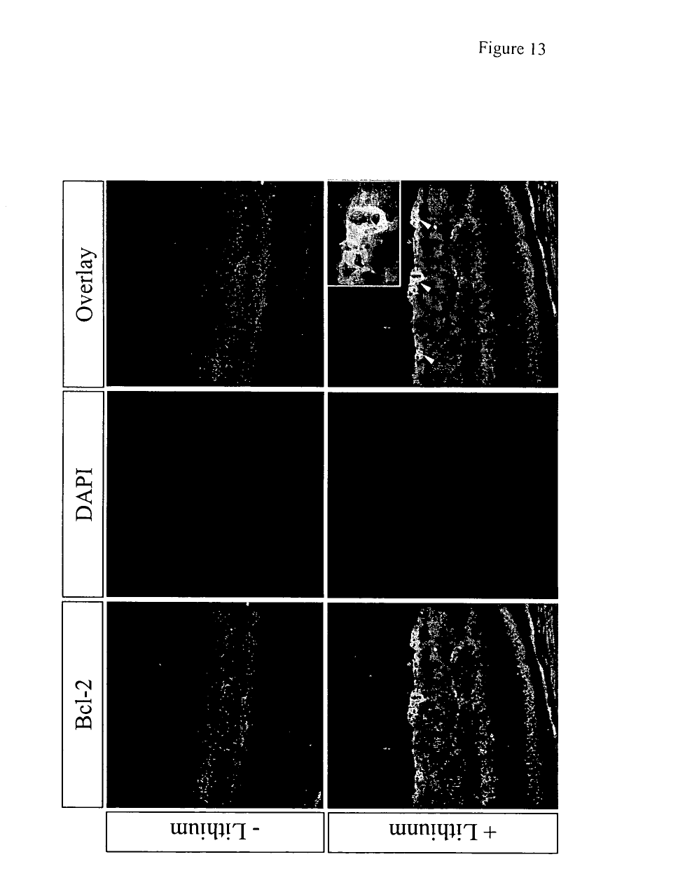

Previously, we have reported that lithium induces Bcl-2 expression in cultured RGCs. To determine whether lithium induces the expression of Bcl-2 in adult retinal neurons in vivo, we fed mice with food chow containing lithium. The serum concentration of lithium measured from mice that consumed a diet containing lithium (n=9) was significant higher (0.80±0.15 mEq/l) than those that consumed a control diet (n=3; <0.1 mEq/l)(FIG. 12A). This concentration is within the well-established safety and therapeutic profile of lithium. The results of quantitative RT-PCR indicated that mice receiving a lithium-containing diet exhibited a strong induction of Bcl-2 mRNA in their retinas compared with mice that received a regular diet, which revealed an undetectable level of Bcl-2 mRNA (FIG. 12B). To determine the type of cells that upregulate Bcl-2 in the mouse retina, we performed immunostaining for Bcl-2 protein. Expression of Bcl-2 protein was detected in the ganglion cell layer of mice that consumed a lithium-containing diet but not in the retinas of mice that consumed a control diet (FIG. 13). Therefore, lithium can induce Bcl-2 expression in adult mouse retina in vivo.

Robust Regeneration of the Optic Nerve Induced by Lithium and Astrotoxin

To evaluate the effects of lithium used simultaneously with astrotoxin in optic nerve regeneration in adult mice, an on optic nerve crush was performed in adult mice. Immediately after the optic nerve crush, a piece of gelform soaked in astrotoxin solution or PBS was placed against the crush site. Thereafter, mice were divided into two groups: one group received a diet containing lithium, and the other received a control diet.

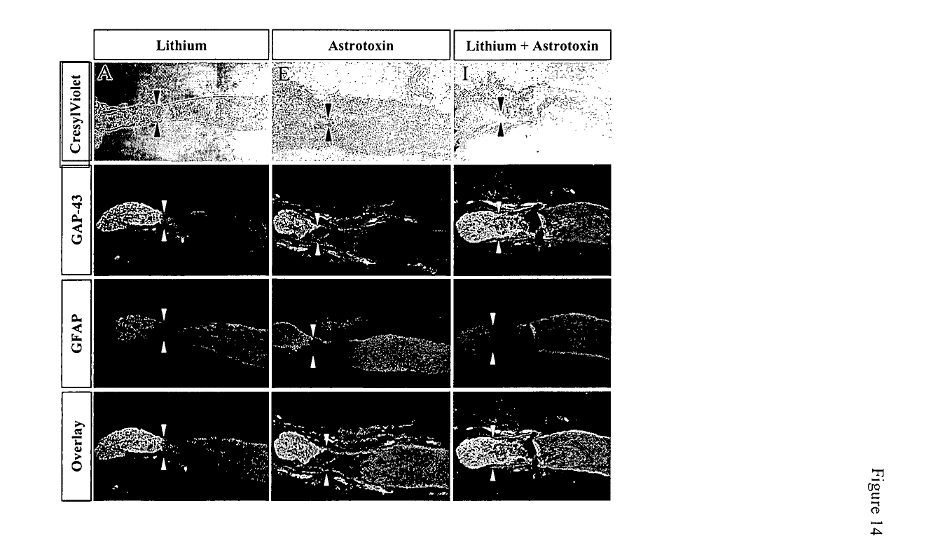

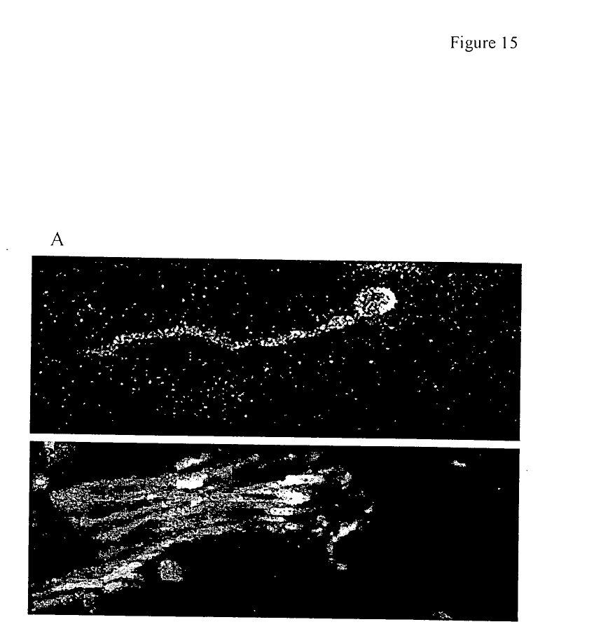

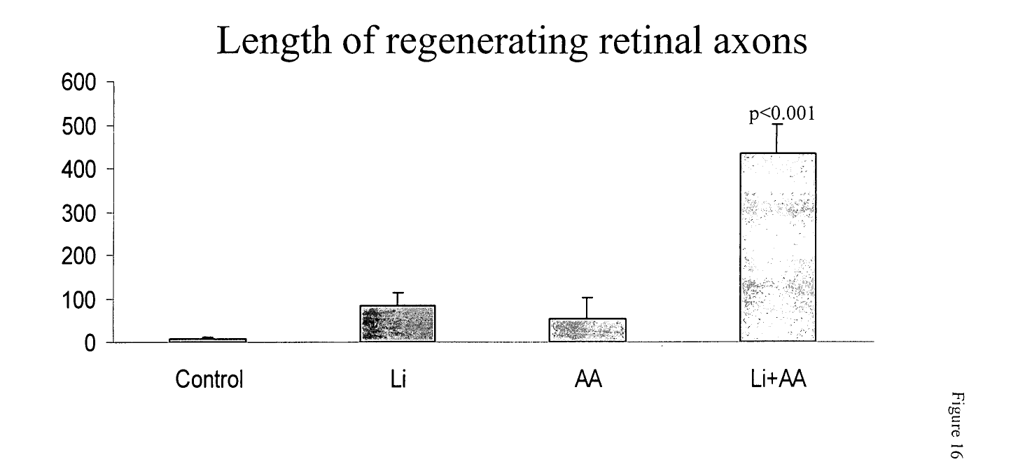

Mice were sacrificed on day 8 after the nerve crush to examine axon regeneration. The crush site of the optic nerve was identified by a dense area of degenerating cells and a narrow outline of the optic nerve following staining of nerve sections with cresyl violet (FIG. 14A, E, I). Regenerating axons were revealed by immunodetection of GAP-43 labeling. No regenerative response was observed in mice that consumed a control diet. In mice receiving a lithium-containing diet alone, without the application of astrotoxin (n=11, FIG. 14A-D), again, no sign of regeneration was observed. Many severed retinal axons expressing GAP-43 were seen to terminate anterior to the crushed site of the severed optic nerve (FIG. 14B). Expression of GFAP, a marker of astrocytes, was detected along the entire nerve (FIG. 14C). Moreover, a similar observation was made in mice with optic nerve crushed and administration of gelfoam soaked with PBS. Similarly, in the group of mice receiving astrotoxin alone (n=3, FIG. 15E-H), no sign of regeneration was observed (FIG. 15F) although a region devoid of immunorective astrocytes (GFAP-negative) was generated posterior to the crushed site (FIG. 15G). However, when the mice received lithium and astrotoxin simultaneously (n=6, FIG. 15I-L), the severed retinal axons re-grew robustly into the GFAP-negative zone of the optic nerve (FIG. 15L). A significant number of growth cone-like structures of regenerating axons were observed posterior to the crushed site (FIG. 16A-C).

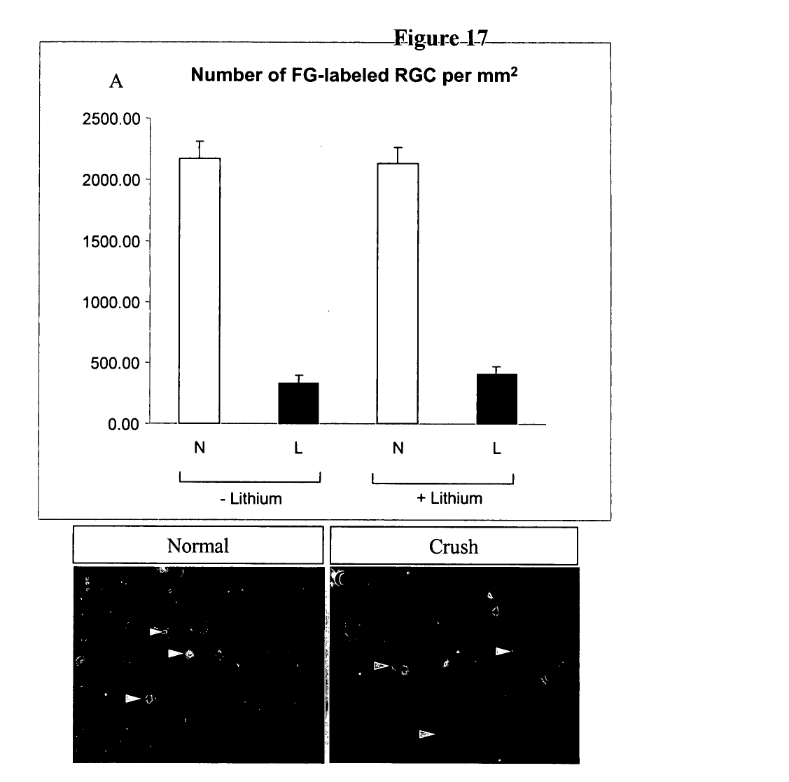

The length of regenerating axons extending from the crushed site of the optic nerve following immunostaining with an anti-GAP-43 antibody was measured: over 400 μm long of regenerating axons were detected in mice simultaneously receiving lithium and astrotoxin. Such regenerated axons were significantly longer than that of the other groups (FIG. 17, p<0.001). Although longer regenerating axons were observed in mice receiving only lithium (80 μm) or only astrotoxin (50 μm) relative to the control (10 μm), there were no significant differences among these groups (p>0.05).

Density of Survival RGCs Following Optic Nerve Crush

The neuroprotective effect of lithium-containing diet on the survival of fluorogold (FG)-labeled RGCs was assessed. Following application of FG in superior colliculus, surviving RGCs showed bright yellow grains in the cytoplasm (FIG. 14B). In normal retina, the density of FG-labeled RGCs is similar in lithium-treated mice (2134/mm²) and control mice (2170/mm²). Following optic nerve crush, the number of surviving RGCs is greatly decreased to 404/mm² in retina of lithium-treated mice and 327/mm² in that of control mice, but there is no significant difference between these 2 groups (FIG. 14A, P>0.05). Although lithium has been reported as a neuroprotective agent in CNS, at least in the present paradigm, lithium-containing diet could not rescue the RGCs from death.

Thus, we have shown that intake of a lithium supplemented diet could induce Bcl-2 expression in the ganglion cell layer of adult mouse retina and that application of astrotoxin could successfully eliminate astrocytes along the optic nerve in vivo. Our data demonstrates that simultaneously administration of lithium and astrotoxin could induce robust optic nerve regeneration. However, astrotoxin or lithium alone could not significantly induce optic nerve regeneration.

Recently, it was shown that in vivo administration of astrotoxin into the CNS caused specific ablation of astrocytes but no adverse effect on the other cell types including oligodendrocytes, microglia and endothelial cells^{9″,11}″. Astrocytic scar²″ and myelins including NogoA^{12″,13}″, MAG^{14″,15}″ and Ompg^{16″,11}″, have been identified as growth inhibitors in CNS. Chondroitin sulfate proteoglycans (CSPG) from astrocytes and other cell type has been shown to block the CNS regeneration^{2″,18}″. Fawcetts and colleagues showed that removal of mixed glial cells population including astrocytes, oligodendrocytes/myelin by a neurotoxic agent, ethidium bromide, could improve some axonal regeneration in the lesioned nigrostriatal tract¹⁹″. We have shown here that an astrocyte-ablated milieu is sufficient to allow axonal regeneration.

Lithium has long been used as a mood-stabilizing drug²⁰″. Recently, other effects of lithium have been uncovered, such as induced expression of Bcl-2 in CNS neurons²¹″, promotion of neurite outgrowth in vitro⁷″, prevention of neuronal lost²²″, and stimulation of the proliferation of neuronal progenitor cells²³″. The present study showed that intake of lithium could elevate the serum lithium level and induced the expression of Bcl-2 in mouse retina, but it has no effect on prevention of RGCs death, at least in our paradigm. The regulation of Bcl-2 expression has been shown through increase in cAMP response element binding protein (CREB) activity in PC12 cells²⁴″. Active glycogen synthase kinase-3 (GSK-3) could suppress the CREB activity. Lithium was shown to inhibit the activity of GSK-3 25,, and reverse the inhibitory effect on CREB activity ²⁶″. Therefore, lithium-induced Bcl-2 expression in mice retina might be related to the activation of CREB though the inhibition of GSK-3 by lithium.

In addition to the well-known anti-apoptotic function of Bcl-2, over-expressing Bcl-2 in neurons has been shown to enhance CNS regeneration in neonatal mice in vivo ⁶″ and in vitro ⁷″ but not in adult.⁸″ Our results showed that either ablation of astrocytes or lithium-induced expression of Bcl-2 in adult retina could not enhance optic nerve regeneration. It suggests that mature neurons may lack intrinsic growth ability to re-grow even in a growth permissive environment and Bcl-2 expressing neurons could not overcome the hostile environment in mature CNS. Therefore, robust optic nerve regeneration could only be achieved in mice receiving concurrent treatment of lithium and astrotoxin. It suggests that Bcl-2-support growth mechanism and removal of astrocytes or inactivation of reactive astrocytes is essential to allow robust axonal regeneration. Astrocytes may play a critical role in preventing CNS regeneration.

Materials and Methods

Surgical procedure: All experimental procedures and use of animals were approved by Animal Care and Use Committee in the Schepens Eye Research Institute and adhered to the ARVO statement for the Use of Animals in Ophthalmic and Vision Research. Adult C57BL/6J mice were anesthetized with a mixture of ketamine (12.5 mg/ml, Sigma) and xylazine (2.5 mg/ml, Sigma) (5 μ/g). Optic nerve was exposed intraorbitally and crushed for 3 seconds using fine forceps. Immediately after nerve crush, a piece of gelfoam soaked with L-α-aminoadipate (10 mg/ml; Sigma) was placed against the crushed site. Gelfoam soaked with phosphate buffered saline (PBS) was used as control. To ensure continued delivery of astrotoxin, the gelfoam was replaced every 3 days post-injury (DPI). Following recovery from the optic nerve surgery, mice were divided into two groups: one group received a diet supplemented with lithium (3.5 mg lithium carbonate/kg, Bio-Serv), and the other group received a control diet. Mice were sacrificed on 8 DPI, and the mouse serum was collected to determine serum lithium concentration (Medtox Laboratories Inc).

Retrograde labeling of RGCs: To study the neuroprotective effect of lithium, RGCs were pre-labeled by placing bilaterally a gelfoam soaked with 6% FluoroGold on the superior colliculus (SC) for 1 week before optic nerve crush. After sacrificing, whole-mount retinas were prepared. At least 12 non-overlapping areas of the retina were selected randomly and photographed. Surviving RGCs that were labeled with FluoroGold were counted, and the density of survival RGCs was determined.

Retinal histology and immunohistochemistry: The eyeball and optic nerve were dissected out, fixed in 4% paraformaldehyde for 1 hr, cryoprotected and embedded in O.T.C. The tissues were then cryosectioned at 12 μm and subjected to cresyl violet staining or immunohistochemistry. For immunofluorescence labeling, tissue sections were reacted with Cy-3 conjugated mouse anti-GFAP (1:1000, Sigma) or rabbit anti-GAP-43 (1:200, Chemicon). The signal of GAP-43 was then visualized by FITC-conjugated goat anti-rabbit IgG (1:250, Vector Lab.). To detect the expression of Bcl-2 protein, the eyeballs were dissected out and embedded without fixation. Before immunostaining, retinal sections were fixed with acetone that was stored at −20° C. for 15 min and incubated with Bcl-2 antibody (1:50, Transduction lab) followed by reaction with biotinylated anti-mouse IgG (1:100, Vector Lab.) and DTAF-conjugated Streptavidin (1:100, Jackson Lab). The sections were then counter-stained with DAPI (1:150, Sigma) to reveal cell nuclei before they were mounted with Vectashield (Vector Lab) and examined under epifluorescence microscope.

Quantitation of Axon Regeneration: Following staining with anti-GAP-43 antibody, the longest distance of axon regeneration axon was measured from the crushed site in optic nerve sections. At least 4 sections were sampled from each optic nerve, and the measured lengths of regenerating axons were averaged. Statistical significance was determined by One-way ANOVA and Tukey-Kramer multiple comparison tests.

Semi-quantitative RT-PCR: The procedure of RT-PCR was described previously ⁷. In brief, total retinal RNAs were extracted from adult wild-type mice that consumed a regular diet or a diet containing lithium. Retinal RNAs isolated from Bcl-2 transgenic mice driven under the promoter of neural specific enolase promoter were used as positive control. To clean off potentially contaminating genomic DNA, RNAs were treated with DNA-free (Ambion) before they were subjected to reverse transcription. The relative amounts of cDNA were normalized by comparing the level of PCR amplification for an internal control gene, glyceraldehyde-3-phosphate dehydrogenase (G3PDH) (forward primer: agaacatcatccctgcatcc; reverse primer: agccgtattcattgtcatacc). Thus, each PCR reaction contained equivalent amounts of cDNAs. The relative amount of target gene, mouse Bcl-2 (forward primer: agcattgcggaggaagtaga; reverse primer: tagcccctctgtgacagctt), was then amplified and compared. PCR products were resolved by electrophoresis using 2% agarose gels and photographed with a Kodak DC 120 digital camera (Eastman Kodak; Rochester, N.Y.).

References

1. B. S. Bregman, Curr. Opin. Neurobiol. 8, 800 (1998).
2. D. F. Chen, S. Tonegawa, Cell Death Differ. 5, 816 (1998).
3. J. L. Goldberg, B. A. Barres, Annu. Rev. Neurosci. 23, 579 (2000).
4. P. J. Homer, F. H. Gage, Nature 407, 963 (2000).
5. A. E. Fournier, S. M. Strittmatter, Curr. Opin. Neurobiol. 11, 89 (2001).
6. D. A. Morgenstern, R. A. Asher, J. W. Fawcett, Prog. Brain Res. 137, 313 (2002).
7. D. F. Chen, S. Jhaveri, G. E. Schneider, Proc. Natl. Acad. Sci. U. S. A. 92, 7287 (1995).
8. J. L. Goldberg, M. P. Klassen, Y. Hua, B. A. Barres, Science 296, 1860 (2002).
9. D. F. Chen, G. E. Schneider, J. C. Martinou, S. Tonegawa, Nature 385, 434 (1997).
10. L. Cristino, A. Pica, F. Della Corte, M. Bentivoglio, Neuroscience 101, 451 (2000).
11. M. Hilton, G. Middleton, A. M. Davies, Bcl-2 influences axonal growth rate in embryonic sensory neurons. Curr. Biol. 7, 798 (1997).
12. S. Chierzi, E. Strettoi, M. C. Cenni, L. Maffei, J. Neurosci. 19, 8367 (1999).
13. C. Lodovichi, G. Di Cristo, M. C. Cenni, L. Maffei, Eur. J. Neurosci. 13, 833 (2001).
14. J. C. Martinou, et al., Neuron 13, 1017 (1994).
15. K. Ohmi, et al., Proc. Natl. Acad. Sci. U. S. A. 100, 1902 (2003).
16. S. Jhaveri, M. A. Edwards, G. E. Schneider, Exp. Brain Res. 87, 371 (1991).
17. J. L. Goldberg, B. A. Barres, Nature 403, 369 (2000).
18. J. Silver, J. Neurol. 242, S22 (1994).
19. S. Jhaveri, R. S. Erzurumlu, B. Friedman, G. E. Schneider, Glia 6, 138 (1992).
20. D. Hunt, M. R. Mason, G. Campbell, R. Coffin, P. N. Anderson, Mol. Cell Neurosci. 20, 537 (2002).
21. A. Josephson, et al., J. Comp. Neurol. 453, 292 (2002).
22. J. M. Vela, B. Gonzalez, B. Castellano, Brain Res. Brain Res. Rev. 26, 29 (1998).
23. D. Dahl, D. C. Rueger, A. Bignami, K. Weber, M. Osborn, Eur. J. Cell Biol. 24, 191 (1981).
24. E. Lazarides, Annu. Rev. Biochem. 51, 219 (1982).
25. M. Khurgel, A. C. Koo, G. O. Ivy, Glia 16, 351 (1996).
26. J. McGraw, G. W. Hiebert, J. D. Steeves, J. Neurosci. Res. 63, 109 (2001).
27. M. Pekny, et al., J. Cell Biol. 145, 503 (1999).
28. R. Kinouchi, et al., Nature Neurosci. in press.
29. S. J. Davies, et al., Nature 390, 680 (1997).
30. S. J. Davies, D. R. Goucher, C. Doller, J. Silver, J Neurosci. 19, 5810 (1999).
1′. Studer, L., Tabar, V. & McKay, R. D. Transplantation of expanded mesencephalic precursors leads to recovery in parkinsonian rats. Nat. Neurosci. 1, 290-5. (1998).
2′. Berson, E. L. & Jakobiec, F. A. Neural retinal cell transplantation: ideal versus reality. Ophthalmology 106, 445-446 (1999).
3′. Dunnett, S.B. Repair of the damaged brain. The Alfred Meyer Memorial Lecture 1998. Neuropathol. Appl. Neurobiol. 25, 351-362 (1999).
4′. McDonald, J. W., et al. Transplanted embryonic stem cells survive, differentiate and promote recovery in injured rat spinal cord. Nat. Med. 5, 1410-1412 (1999).
5′. Svendsen, C. N., Bhattacharyya, A. & Tai, Y. T. Neurons from stem cells: preventing an identity crisis. Nat. Rev. Neurosci. 2, 831-834 (2001).
6′. Takahashi, M., Palmer, T. D., Takahashi, J. & Gage, F. H. Widespread integration and survival of adult-derived neural progenitor cells in the developing optic retina. Mol. Cell Neurosci. 12, 340-348 (1998).
7′. Young, M. J., Ray, J., Whiteley, S. J., Klassen, H. & Gage, F. H. Neuronal differentiation and morphological integration of hippocampal progenitor cells transplanted to the retina of immature and mature dystrophic rats. Mol. Cell Neurosci. 16, 197-205 (2000).
8′. Lu, B., et al. Transplantation of EGF-responsive neurospheres from GFP transgenic mice into the eyes of rd mice. Brain Res. 943, 292-300 (2002).
9′. Gouras, P., Du, J., Kjeldbye, H., Yamamoto, S. & Zack, D. J. Long-term photoreceptor transplants in dystrophic and normal mouse retina. Invest. Ophthalmol. Vis. Sci. 35, 3145-3153 (1994).
10′. Zhang, Y., Amer, K., Ehinger, B. & Perez, M. T. Limitation of anatomical integration between subretinal transplants and the host retina. Invest. Ophthalmol. Vis. Sci. 44, 324-331 (2003).
11′. Seiler, M. & Turner, J. E. The activities of host and graft glial cells following retinal transplantation into the lesioned adult rat eye: developmental expression of glial markers. Brain Res. 471, 111-122 (1988).
12′. Ghosh, F. & Wasselius, J. Muller cells in allogeneic adult rabbit retinal transplants. Glia 40, 78-84 (2002).
13′. Fawcett, J. W. & Asher, R. A. The glial scar and central nervous system repair. Brain Res. Bull. 49, 377-391 (1999).
14′. Eliasson, C., et al. Intermediate filament protein partnership in astrocytes. J. Biol. Chem. 274, 23996-24006 (1999).
15′. Barker, R. A., Dunnett, S. B., Faissner, A. & Fawcett, J. W. The time course of loss of dopaminergic neurons and the gliotic reaction surrounding grafts of embryonic mesencephalon to the striatum. Exp. Neurol. 141, 79-93 (1996).
16′. McGraw, J., Hiebert, G. W. & Steeves, J. D. Modulating astrogliosis after neurotrauma. J. Neurosci. Res. 63, 109-115 (2001).
17′. Pekny, M. Astrocytic intermediate filaments: lessons from GFAP and vimentin knock-out mice. Prog. Brain Res. 132, 23-30 (2001).
18′. Pekny, M., et al. Abnormal reaction to central nervous system injury in mice lacking glial fibrillary acidic protein and vimentin. J. Cell Biol. 145, 503-514 (1999).
19′. Pekny, M., et al. Mice lacking glial fibrillary acidic protein display astrocytes devoid of intermediate filaments but develop and reproduce normally. EMBO J. 14, 1590-1598 (1995).
20′. Gomi, H., et al. Mice devoid of the glial fibrillary acidic protein develop normally and are susceptible to scrapie prions. Neuron 14, 29-41 (1995).
21′. McCall, M. A., et al. Targeted deletion in astrocyte intermediate filament (GFAP) alters neuronal physiology. Proc. Natl. Acad. Sci. USA 93, 6361-6366 (1996).
22′. Colucci-Guyon, E., et al. Mice lacking vimentin develop and reproduce without an obvious phenotype. Cell 79, 679-694 (1994).
23′. Menet, V., et al. Inactivation of the glial fibrillary acidic protein gene, but not that of vimentin, improves neuronal survival and neurite growth by modifying adhesion molecule expression. J. Neurosci. 21, 6147-6158 (2001).
24′. Okabe, M., Ikawa, M., Kominami, K., Nakanishi, T. & Nishimune, Y. ‘Green mice’ as a source of ubiquitous green cells. FEBS Lett. 407, 313-319 (1997).
25′. Smith-Thomas, L. C., et al. An inhibitor of neurite outgrowth produced by astrocytes. J. Cell Sci. 107, 1687-1695 (1994).
26′. McKeon, R. J., Schreiber, R. C., Rudge, J. S. & Silver, J. Reduction of neurite outgrowth in a model of glial scarring following CNS injury is correlated with the expression of inhibitory molecules on reactive astrocytes. J. Neurosci. 11, 3398-3411 (1991).
27′. Cepko, C. L., Austin, C. P., Yang, X., Alexiades, M. & Ezzeddine, D. Cell fate determination in the vertebrate retina. Proc. Natl. Acad. Sci. USA 93, 589-595 (1996).
28′. Young, R. W. Cell differentiation in the retina of the mouse. Anat. Rec. 212, 199-205 (1985).
29′. Gan, L., Xiang, M., Zhou, L., Wagner, D. S., Klein, W. H., Nathans, J. POU domain factor Bm-3b is required for the development of a large set of retinal ganglion cells. Proc. Natl. Acad. Sci. USA 93, 3920-3925 (1996).
30′. Barnstable, C. J. & Drager, U. C. Thy-1 antigen: a ganglion cell specific marker in rodent retina. Neuroscience 11, 847-855 (1984).
31′. Pekny, M., et al. GFAP-deficient astrocytes are capable of stellation in vitro when cocultured with neurons and exhibit a reduced amount of intermediate filaments and an increased cell saturation density. Exp. Cell Res. 239, 332-343 (1998).
32′. Van Hoffelen, S. J., Young, M. J., Shatos, M. A. & Sakaguchi, D. S. Incorporation of murine brain progenitor cells into the developing mammalian retina. Invest. Ophthalmol. Vis. Sci. 44, 426-434 (2003).
33′. Nishida, A., et al. Incorporation and differentiation of hippocampus-derived neural stem cells transplanted in injured adult rat retina. Invest. Ophthalmol. Vis. Sci. 41, 4268-4274 (2000).
34′. Kurimoto, Y., et al. Transplantation of adult rat hippocampus-derived neural stem cells into retina injured by transient ischemia. Neurosci Lett. 306, 57-60 (2001).
35′. Qiu, J., Cai, D. & Filbin, M. T. Glial inhibition of nerve regeneration in the mature mammalian CNS. Glia 29, 166-174 (2000).
36′. Fournier, A. E. & Strittmatter, S. M. Repulsive factors and axon regeneration in the CNS. Curr. Opin. Neurobiol. 11, 89-94 (2001).
37′. Xu, K., Malouf, A. T., Messing, A. & Silver, J. Glial fibrillary acidic protein is necessary for mature astrocytes to react to beta-amyloid. Glia 25, 390-403 (1999).
38′. Menet, V., Gimenez, Y. R. M., Sandillon, F. & Privat, A. GFAP null astrocytes are a favorable substrate for neuronal survival and neurite growth. Glia 31, 267-272 (2000).
39′. Wang, X., Messing, A. & David, S. Axonal and nonneuronal cell responses to spinal cord injury in mice lacking glial fibrillary acidic protein. Exp. Neurol. 148, 568-576 (1997).
1″. Asher R A, Morgenstern D A, Moon L D, et al. Chondroitin sulphate proteoglycans: inhibitory components ofthe glial scar. Prog Brain Res 2001;132:611-619.
2″. Filbin M T. Myelin-associated inhibitors of axonal regeneration in the adult mammalian CNS. Nat Rev Neurosci 2003;4:703-713.
3″. Yick L W, Wu W, So K F, et al. Chondroitinase ABC promotes axonal regeneration of Clarke's neurons after spinal cord injury. Neuroreport 2000; 11:1063-1067.
4″. Hunt D, Coffin R S, Anderson P N. The Nogo receptor, its ligands and axonal regeneration in the spinal cord; a review. J Neurocytol 2002;31 :93-120.
5″. Chen D F, Jhaveri S, Schneider G E. Intrinsic changes in developing retinal neurons result in regenerative failure of their axons. Proc Natl Acad Sci USA 1995;92:7287-7291.
6″. Chen D F, Schneider G E, Martinou J C, et al. Bcl-2 promotes regeneration of severed axons in mammalian CNS. Nature 1997;385:434-439.
7″. Huang X, Wu DY, Chen G, et al. Support of retinal ganglion cell survival and axon regeneration by lithium through a Bcl-2-dependent mechanism. Invest Ophthalmol Vis Sci 2003;44:347-354.
8″. Inoue T, Hosokawa M, Morigiwa K, et al. Bcl-2 overexpression does not enhance in vivo axonal regeneration of retinal ganglion cells after peripheral nerve transplantation in adult mice. J Neurosci 2002;22:4468-4477.
9″. Takada M, Hattori T. Fine structural changes in the rat brain after local injections of gliotoxin, alpha-aminoadipic acid. Histol Histopathol 1986; 1:271-275.
10″. Khurgel M, Koo A C, Ivy G O. Selective ablation of astrocytes by intracerebral injections of alpha-aminoadipate. Glia 1996;16:351-358.
11″. McGraw J, Hiebert G W, Steeves J D. Modulating astrogliosis after neurotrauma. J Neurosci Res 2001;63:109-115.
12″. Chen M S, Huber A B, van der Haar M E, et al. Nogo-A is a myelin-associated neurite outgrowth inhibitor and an antigen for monoclonal antibody IN-1. Nature 2000;403:434-439.
13″. GrandPre T, Nakamura F, Vartanian T, et al. Identification of the Nogo inhibitor of axon regeneration as a Reticulon protein. Nature 2000;403:439-444.
14″. McKerracher L, David S, Jackson D L, et al. Identification of myelin-associated glycoprotein as a major myelin-derived inhibitor of neurite growth. Neuron 1994;13:805-811.
15″. Mukhopadhyay G, Doherty P, Walsh F S, et al. A novel role for myelin-associated glycoprotein as an inhibitor of axonal regeneration. Neuron 1994;13:757-767.
16″. Kottis V, Thibault P, Mikol D, et al. Oligodendrocyte-myelin glycoprotein (OMgp) is an inhibitor of neurite outgrowth. J Neurochem 2002;82:1566-1569.
17″. Wang K C, Koprivica V, Kim J A, et al. Oligodendrocyte-myelin glycoprotein is a Nogo receptor ligand that inhibits neurite outgrowth. Nature 2002;417:941-944.
18″. Jones L L, Yamaguchi Y, Stallcup W B, et al. NG2 is a major chondroitin sulfate proteoglycan produced after spinal cord injury and is expressed by macrophages and oligodendrocyte progenitors. J Neurosci 2002;22:2792-2803.
19″. Moon L D, Brecknell J E, Franklin R J, et al. Robust regeneration of CNS axons through a track depleted of CNS glia. Exp Neurol 2000; 161:49-66.
20″. Pilcher H R. Drug research: the ups and downs of lithium. Nature 2003;425:118-120.
21″. Chen G, Zeng W Z, Yuan P X, et al. The mood-stabilizing agents lithium and valproate robustly increase the levels of the neuroprotective protein bcl-2 in the CNS. J Neurochem 1999;72:879-882.
22″. Hashimoto R, Takei N, Shimazu K, et al. Lithium induces brain-derived neurotrophic factor and activates TrkB in rodent cortical neurons: an essential step for neuroprotection against glutamate excitotoxicity. Neuropharmacology 2002;43:1173-1179.
23″. Hashimoto R, Senatorov V, Kanai H, et al. Lithium stimulates progenitor proliferation in cultured brain neurons. Neuroscience 2003;117:55-61.
24″. Pugazhenthi S, Nesterova A, Sable C, et al. Akt/protein kinase B up-regulates Bcl-2 expression through cAMP-response element-binding protein. J Biol Chem 2000;275:10761-10766.
25″. Jope R S. Lithium and GSK-3: one inhibitor, two inhibitory actions, multiple outcomes. Trends Pharmacol Sci 2003;24:441-443.
26″. Ozaki N, Chuang D M. Lithium increases transcription factor binding to AP-1 and cyclic AMP-responsive element in cultured neurons and rat brain. J Neurochem 1997;69:2336-2344.
   Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

Equivalents

While specific embodiments of the methods and compositions presented herein have been discussed, the above specification is illustrative and not restrictive. Many variations of the methods and compositions presented herein will become apparent to those skilled in the art upon review of this specification and the claims below. The full scope of the methods and compositions presented herein should be determined by reference to the claims, along with their full scope of equivalents, and the specification, along with such variations.

1. A composition comprising an agent that inhibits glial scar formation and an agent that increases bcl-2 activity or protein levels.

2. The composition of claim 1, wherein the agent that inhibits glial scar formation is astrotoxin or an analog thereof.

3. The composition of claim 1, wherein the agent that increases bcl-2 activity or protein levels is lithium or a salt thereof.

4. The composition of claim 3, wherein the agent that inhibits glial scar formation is astrotoxin or an analog thereof.

5. A kit comprising an agent that inhibits glial scar formation and an agent that increases bcl-2 activity or protein levels.

6. The kit of claim 5, wherein the agent that inhibits glial scar formation is astrotoxin or an analog thereof.

7. The kit of claim 5, wherein the agent that increases bcl-2 activity or protein levels is lithium or a salt thereof.

8. The kit of claim 7, wherein the agent that inhibits glial scar formation is astrotoxin or an analog thereof.

9. The kit of claim 7, wherein the lithium is in a composition for oral administration.

10. A method for promoting a permissive environment for axon regeneration or cell transplantation in a mammalian tissue that comprises astroglial cells, comprising inhibiting glial scar formation in the tissue.

11. The method of claim 10, wherein inhibiting glial scar formation comprises contacting the mammalian tissue with astrotoxin or an analog thereof.

12. The method of claim 10, wherein inhibiting glial scar formation comprises inhibiting the expression of glial fibrillary acid protein (GFAP) and vimentin (Vim) in astroglial cells.

13. The method of claim 12, wherein the mammalian tissue is in a mammalian subject and the method comprises administering to the subject at least one agent that inhibits glial scar formation.

14. The method of claim 13, wherein the mammalian tissue is an ocular tissue.

15. The method of claim 14, wherein the mammalian tissue is the central nervous system (CNS).

16. A method for promoting the regeneration of an axon of a neural cell in a mammalian tissue comprising astroglial cells, comprising (i) inhibiting glial scar formation in the tissue and (ii) increasing bcl-2 activity or protein levels in the neural cell.

17. The method of claim 16, wherein inhibiting glial scar formation comprises contacting the tissue with astrotoxin or an analog thereof.

18. The method of claim 16, wherein inhibiting glial scar formation comprises inhibiting the expression of GFAP and VIM in astroglial cells of the tissue.

19. The method of claim 16, wherein increasing bcl-2 activity or protein level in the neural cell comprises contacting the neural cell with lithium or an analog thereof.

20. The method of claim 16, further contacting the mammalian tissue with at least one neuron stimulating factor.

21. The method of claim 20, wherein the at least one neuron stimulating factor is selected from the group consisting of fibroblast growth factor, ciliary neurotrophic factor, nerve growth factor, and brain-derived neurotrophic factor.

22. The method of claim 16, wherein the mammalian tissue is in a mammalian subject and the method comprises administering to the subject an agent that inhibits glial scar formation and an agent that increases bcl-2 activity or protein levels in the neural cell.

23. The method of claim 22, wherein the agent that increases bcl-2 activity or protein levels is lithium or a salt thereof.

24. The method of claim 23, wherein the agent that inhibits glial scar formation is administered into the tissue and the lithium or salt thereof is administered orally, intranasally, intravenously, intramuscularly or topically to the mammalian subject.

25. The method of claim 24, wherein the lithium or salt thereof is administered orally.

26. The method of claim 16, wherein the mammalian tissue is an ocular tissue.

27. The method of claim 16, wherein the mammalian tissue is the CNS.

28. The method of claim 22, wherein the subject is a human, a domesticated animal, a livestock animal, a cow, a dog, a cat, a goat or a mouse.

29. The method of claim 28, wherein the subject is a human.

30. A method for treating a condition in which axons have been severed in a tissue of a subject, comprising administering to the subject an agent that inhibits glial scar formation in the tissue and an agent that increases bcl-2 activity or protein levels.

31. The method of claim 30, wherein the agent that inhibits glial scar formation is astrotoxin or an analog thereof and the agent that increases bcl-2 is lithium or a salt thereof.

32. The method of claim 31, wherein astrotoxin or the analog thereof is administered into the tissue and lithium or the salt thereof is administered orally to the subject.

33. A method for introducing a neural cell into a neural tissue of a mammalian subject comprising (i) administering to a mammalian subject an agent that inhibits glial scar formation in a neural tissue of the subject; and (ii) introducing a neural cell into the neural tissue of the subject.

34. The method of claim 33, wherein the agent is astrotoxin or an analog thereof.

35. The method of claim 33, further comprising administering an agent that increases bcl-2 activity or protein levels in the neuron.

36. The method of claim 35, wherein the agent that increases bcl-2 activity or protein levels is lithium or a salt thereof.

37. The method of claim 36, wherein lithium or a salt thereof is administered orally, intranasally, intravenously, intramuscularly or topically to the mammalian subject.

38. The method of claim 37, wherein the lithium or salt thereof is administered orally.

39. The method of claim 33, wherein the neural tissue is an ocular tissue.

40. The method of claim 33, wherein the neural tissue is the CNS.

41. The method of claim 33, wherein the subject is a human, a domesticated animal, a livestock animal, a cow, a dog, a cat, a goat or a mouse.

42. The method of claim 41, wherein the subject is a human.

43. An assay for identifying an agent that promotes a permissive environment for axon regeneration or cell transplantation comprising (i) contacting a reactive astroglial cell with a test agent; and (ii) determining the effect of the test agent on the activity of the astroglial cell, wherein a lower activity in the presence of the test agent indicates that the agent promotes a permissive environment for axon regeneration or cell transplantation.

44. The assay according to claim 43, comprising determining the effect of the agent on the activity or protein level of GFAP and VIM.

45. The assay of claim 44, comprising determining whether the test agent inhibits glial scar formation.

44. An assay for identifying an agent that promotes a permissive environment for axon regeneration or cell transplantation, comprising (i) contacting a tissue or cells comprising a reactive astroglial cell with a test agent and an agent that stimulates axon regeneration; and (ii) determining whether axon regeneration has occurred, wherein the presence of axon regeneration in the tissue or cells contacted with a test agent relative to that not contacted with a test agent indicates that the test agent is an agent that promotes a permissive environment for axon regeneration or cell transplantation.

45. An assay for identifying an agent that promotes axon regeneration, comprising (i) contacting a tissue or cells comprising a neural cell having a severed axon and and astroglial cells with a test agent and an agent that provides a permissive environment for axonal regeneration; and (ii) determining whether axon regeneration has occurred, wherein the presence of axon regeneration in the tissue or cells contacted with a test agent relative to that not contacted with a test agent indicates that the test agent is an agent that promotes axon regeneration.
